(19) United States
(12) Patent Application Publication
(10) Pub. No.: US 2026/0295198 A1
(43) Pub. Date: Oct. 1, 2026

(54) SYNCHRONIZED AUDIOVISUAL NEUROSTIMULATION DEVICES, SYSTEMS AND METHODS

(71) Applicant: BrainTap, Inc., New Bern, NC (US)

(72) Inventors: Patrick K. Porter, New Bern, NC (US); Francisco J. Cidral Filho, Windermere, FL (US); Matteo Karim Carlo Edmondo Borri, San Rafael, CA (US); Kenneth Tarlow, San Rafael, CA (US); Emiliano Amarante Veiga, Chapeco-SC (BR)

(21) Appl. No.: 19/094,339

(22) Filed: Mar. 28, 2025

(51) Int. Cl. A61M 21/00; A61N 5/06

(52) U.S. Cl. CPC A61M 21/00; A61N 5/0622; A61M 2021/0027; A61M 2021/0044; A61N 2005/0626; A61N 2005/0648; A61N 2005/0652; A61N 2005/0659; A61N 2005/0663

(57) ABSTRACT

The present disclosure provides a neurostimulation system comprising a headset, visor, and optional helmet controlled by a controller. The headset delivers audio signals including binaural beats and isochronic tones in a range from 18 to 0.5 Hz. The visor delivers visual signals via light-emitting diodes emitting light at 470 nanometers flickering at 18 to 0.5 Hz. The headset and or visor or combination thereof delivers transcranial photobiomodulation via light at 660 nm and 850 nm wavelengths with a total irradiance of 1000 mW/cm2 per minute. The controller synchronizes and controls delivery of the audio signals, visual signals, and photobiomodulation to alter brainwave activity in a user.

### CROSS-REFERENCE TO RELATED APPLICATIONS

This application relates to U.S. application Ser. No. 16/665,213, titled “Auriculotherapy Apparatus and System and Methods of Use Thereof”, filed Oct. 28, 2019, which is hereby incorporated by reference in its entirety.

### FIELD OF INVENTION

The present disclosure relates to neurostimulation and brainwave entrainment systems, and more particularly to devices, systems and methods for stimulating and synchronizing brainwaves using encoded light and audio signals.

### BACKGROUND

Neurostimulation and brainwave entrainment techniques have gained increasing attention in recent years as potential methods for influencing cognitive function and mental states. These approaches aim to modulate brain activity through external stimuli, typically in the form of audio and visual signals.

Brainwave entrainment refers to the capacity of the brain to naturally synchronize its brainwave frequencies with the rhythm of external stimuli. This phenomenon, also known as the frequency following response, has been observed and studied for decades. Various forms of brainwave entrainment have been developed, including binaural beats, isochronic tones, and visual stimulation techniques.

While traditional meditation and relaxation practices have long been used to alter mental states, technological advancements have enabled the development of more targeted methods of influencing brain activity. These modern approaches often combine multiple stimuli, such as synchronized light and sound patterns, to create immersive experiences designed to induce specific brainwave states.

The potential applications of neurostimulation and brainwave entrainment are diverse, ranging from stress reduction and improved sleep quality to enhanced focus and creativity. However, the effectiveness of these techniques can vary between individuals, and there remains a need for more comprehensive approaches to brain stimulation.

Despite the growing interest in these fields, many existing systems for neurostimulation and brainwave entrainment face limitations. These may include a lack of adaptability to individual user needs, insufficient integration of multiple stimulation modalities, and challenges in providing accurate, real-time feedback on the user's brain state.

As research in neuroscience and cognitive enhancement continues to advance, there is an ongoing need for improved systems and methods that can effectively modulate brain activity to achieve desired mental states and cognitive outcomes.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to at least one aspect of the present disclosure, a kit is provided. The kit includes a headset, and/or visor, and optionally a helmet that combines utilization of Brainwave Entrainment (BWE) and Transcranial Photobiomodulation (tPBM) for altering brainwave power. The kit has an ability to deliver 20-minute sessions with said headset, visor and helmet that produces Binaural beats and Isochronic Tones in a range from 18 to 0.5 Hz together with visual Entrainment via said visor that utilizes light-emitting diode lights at 470 nanometers (nm) that flicker within a range of 18 to 0.5 Hz combined with tPBM delivered via a helmet in 10-minute sessions that transmits 660 nm (n=100) and 850 nm (n=100 ) wavelength light distributed homogenously to each subject so that a total irradiance delivered per session is 1000 mW/cm2 per minute.

According to other aspects of the present disclosure, the kit may include one or more of the following features. The brainwave power may be measured with the Emotiv Epoc+ 14-Channel Wireless electroencephalogram (EEG) Headset. EEG sessions may be conducted at a baseline and wherein individuals receive said EEG sessions over at least a 6 week period with readings conducted for both with 2-minute eyes opened immediately followed by 2-minute eyes closed sessions so that brainwave power of 14 channels can be recorded.

More specifically the present disclosure describes one or more neurostimulation device(s) and or set of devices comprising:
a headset configured to deliver audio signals including binaural beats and isochronic tones in a frequency range from 18 to 0.5 Hz;
a visor configured to deliver visual signals via light-emitting diodes emitting light at 470 nanometers and flickering at frequencies between 18 and 0.5 Hz;
a helmet configured to deliver transcranial photobiomodulation via light at 660 nm and 850 nm wavelengths with a total irradiance of 1000 mW/cm2 per minute; and
a controller operatively connected to the headset, visor, and helmet, said controller configured to synchronize and control delivery of the audio signals, visual signals, and photobiomodulation to alter brainwave activity in a user.

The controller is configured to adjust a frequency of the audio signals and visual signals dynamically during a therapy session, and transition said frequency from higher to lower frequencies over a course of a therapy session.

If a helmet is provided it also comprises a plurality of light-emitting diode arrays distributed across its inner surface to provide homogeneous light distribution, with 100 LEDs emitting light at 660 nm and 100 LEDs emitting light at 850 nm.

The neurostimulation device(s) may further comprise an electroencephalogram (EEG) measurement device configured to measure brainwave activity before and after a neurostimulation therapy session. The controller is configured to adjust delivery of audio signals, visual signals, and photobiomodulation based on feedback from said EEG measurement device. The present disclosure also describes a method comprising:
providing a headset, visor combination that could optionally include a helmet for a user; delivering a Brainwave Entrainment (BWE) session by generating audio signals including binaural beats and isochronic tones in a frequency range from 18 to 0.5 Hz through said headset and activating visual signals via light-emitting diodes emitting light at 470 nanometers and flickering at frequencies between 18 and 0.5 Hz through the visor;
delivering a Transcranial Photobiomodulation (tPBM) session by transmitting light at 660 nm and 850 nm wavelengths through the helmet with a total irradiance of 1000 mW/cm 2 per minute; and synchronizing the delivery of the BWE session and the tPBM session to alter brainwave activity in one or more users.

The method includes a BWE session that lasts for 20 minutes and said tPBM session lasts for 10 minutes, repeated daily over a 6-week intervention period. The method also includes conducting an electroencephalogram (EEG) assessment before and after the intervention period to measure changes in brainwave activity, comprising a 2-minute eyes opened measurement followed by a 2-minute eyes closed measurement across 14 channels corresponding to different regions of a human brain.

The present disclosure also includes a circuit for detecting audio frequencies and activating light channels, comprising: a plurality of frequency detectors, each configured to detect a distinct near-ultrasonic frequency within a range of 18,000 to 20,000 Hz;
a Bluetooth module configured to receive an audio signal;
a preamplifier coupled to the Bluetooth module for signal conditioning; and
a plurality of light activation channels, each triggered by an output of a corresponding frequency detector.

The circuit includes three LM567 integrated circuits, each configured to detect a distinct near-ultrasonic frequency, with unique combinations of passive components to tune frequency response and bandwidth. The capacitors connected to said LM567 integrated circuits are oversized by a factor of approximately 2.1 compared to conventional designs. The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIGS. 1A and 1B illustrates a set of bar charts showing the effects of Audiovisual Brain Entrainment on metabolic score and response time.

FIG. 2 is a graph illustrating the effects of Audiovisual Brain Entrainment on Quantitative EEG scores.

FIGS. 3A-3D are graphs illustrating the effects of audiovisual brain entrainment on cognitive and emotional scores.

FIG. 4 illustrates a graph depicting changes in EEG power across different brainwave frequencies and power following neurostimulation therapy.

FIG. 5 illustrates plots comparing the effects of OLAVE on sleep parameters against a control group over time.

FIG. 6 is a bar chart graph illustrating the effect of audiovisual Brainwave Entrainment on weight reduction.

FIG. 7 is a set of bar charts that illustrate the effect of audiovisual Brainwave Entrainment on Stress, Heart Rate, and Low Frequency to High Frequency ratios

FIG. 8 illustrates bar charts comparing the effects of the H.E.A.L.T.H.Y. H.A.B.I.T.S. program alone and in combination with Brainwave Entrainment on PQSI, POMS, PSS, and GAD-7 scores over a 13-week period.

FIG. 9 illustrates a set of plots showing the effects of a 13-week program on POMS subscales comparing HH and HH+BWE conditions.

FIG. 10 illustrates a set of plots comparing the effects of the H.E.A.L.T.H.Y. H.A.B.I.T.S. program either alone (HH) or in combination with Brainwave Entrainment (HH+BWE) on on mood over a 13-week period.

FIG. 11 illustrates a set of plots showing the effects of PPM combined with ABWE on sleep plus various psychological measures over time using ANOVA.

FIG. 12. also illustrates a set of plots showing the effects of PPM combined with ABWE on various psychological measures over time.

FIG. 13 illustrates a set of bar charts comparing symptom reductions between a control group and a BWE+CT treatment group.

FIG. 14 illustrates a set of bar charts comparing concussion-related symptoms between a control group and a BWE+CT treatment group.

FIG. 15 illustrates a set of bar charts comparing symptom reductions between a control group and a BWE+CT treatment group including NormaTec Pulse 2.0 therapy.

FIG. 16 illustrates a set of bar charts comparing emotional symptoms of emotional stability and sadness between a control group and a BWE+CT treatment group.

FIGS. 17A-17H illustrates a series of bar charts showing Pittsburgh Quality of Sleep Index (PQSI) scores across different components over time.

FIGS. 18A and 18B illustrates comparing bar charts for Perceived Stress Scale (PSS) and Brief Resilience Scale (BRS) scores over time.

FIGS. 19A-19H illustrate a series of bar charts for Profile of Mood States (POMS) scores across various parameters over time.

FIGS. 20A-20F illustrate a series of bar charts showing self-reported measurements on a 10-point Likert scale across multiple time points.

FIGS. 21A-21D illustrate a set of bar charts comparing physiological measurements before and after intervention, including resting heart rate, HRV, SpO2, and respiratory rate.

FIGS. 22A-22C illustrate a set of bar charts comparing baseline and intervention measurements for arterial elasticity, peripheral elasticity, and arterial age.

FIGS. 23A-23D illustrate bar charts comparing sleep-related measurements between baseline and intervention conditions.

FIGS. 24A-24D illustrate bar charts comparing sleep-related measurements between baseline and intervention conditions.

FIGS. 25A-25C illustrate bar charts showing adherence to Braintapping during the intervention phase.

FIG. 26 illustrates bar charts comparing Hamilton Rating for Depression (HAM-D) scores between placebo and BrainTap groups over time.

FIG. 27 illustrates bar charts comparing Pittsburgh Quality of Sleep (PQSI) scores between placebo and BrainTap groups over time.

FIG. 28 illustrates bar charts comparing Resilience Scale and Perceived Stress scores between placebo and BrainTap groups over time.

FIG. 29 illustrates bar charts comparing State-Trait Anxiety Inventory (STAI) scores between placebo and BrainTap groups over time.

FIG. 30 illustrates bar charts comparing SF-36 scores for functional capacity and physical limitations aspects between placebo and BrainTap groups over time.

FIG. 31 illustrates bar charts comparing SF-36 scores for pain and general health states between placebo and BrainTap groups over time.

FIG. 32 illustrates bar charts comparing SF-36 scores for vitality and social aspects between placebo and BrainTap groups over time.

FIG. 33 illustrates bar charts comparing limitations due to emotional aspects and mental health scores from the SF-36 survey between placebo and BrainTap groups over time.

FIG. 34 illustrates a plot and bar chart comparing emotional limitation scores between control and VAS groups and PAIN over time.

FIG. 35 illustrates a plot comparing emotional limitation scores between control and AVS groups over time.

FIG. 36 illustrates a plot comparing alpha activity scores between left and right brain hemispheres over time.

FIG. 37 illustrates bar charts comparing pre- and post-intervention scores for DASS-21 assessments.

FIG. 38 illustrates bar charts comparing pre- and post-intervention scores for POMS assessments.

FIG. 39 illustrates bar graphs comparing PQSI component scores before and after intervention.

FIG. 40 illustrates a perspective view of the neurostimulation device showcasing the integration of audio and visual components within a wearable form factor.

FIG. 41 illustrates a perspective view of a neurostimulation device showcasing the integration of audio and visual components.

FIG. 42 depicts a flowchart according to aspects of the present disclosure that provide the headset visor and helmet kit to a user and helps the user benefit from the neurostimulation.

FIG. 43 shows a circuit diagram of an exemplary LED board, according to at least one embodiment of the present disclosure.

### DETAILED DESCRIPTION

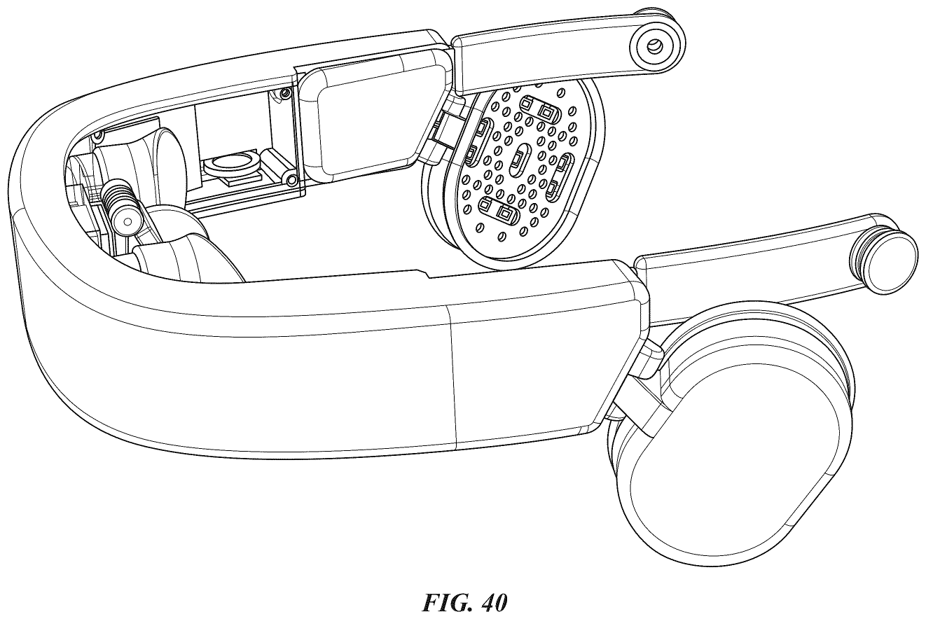

The neurostimulation system comprises a headset, and a visor, wherein the headset and visor can be a singular unit; and an optional helmet that work in combination to deliver specific audio and visual stimuli. As shown in FIG. 40, the system includes a curved headband structure that extends around the head, with integrated optical components positioned on both sides. The headband incorporates a visor portion that extends across the front and is designed to cover the user's eyes.

The headset may provide audio signals such as binaural beats or isochronic tones. In some cases, the headset includes headphone components attached to the sides of the headband, as depicted in FIG. 40. The headphones may feature circular ear cups connected to the main structure through adjustable mounting points.

The visor may emit pulsed light at particular frequencies and wavelengths. As illustrated in FIG. 40, the visor section includes circular housings integrated into the headband structure. These housings may contain optical components that can be used for delivering visual signals. The optical components may be arranged in a symmetrical configuration on both sides of the headband.

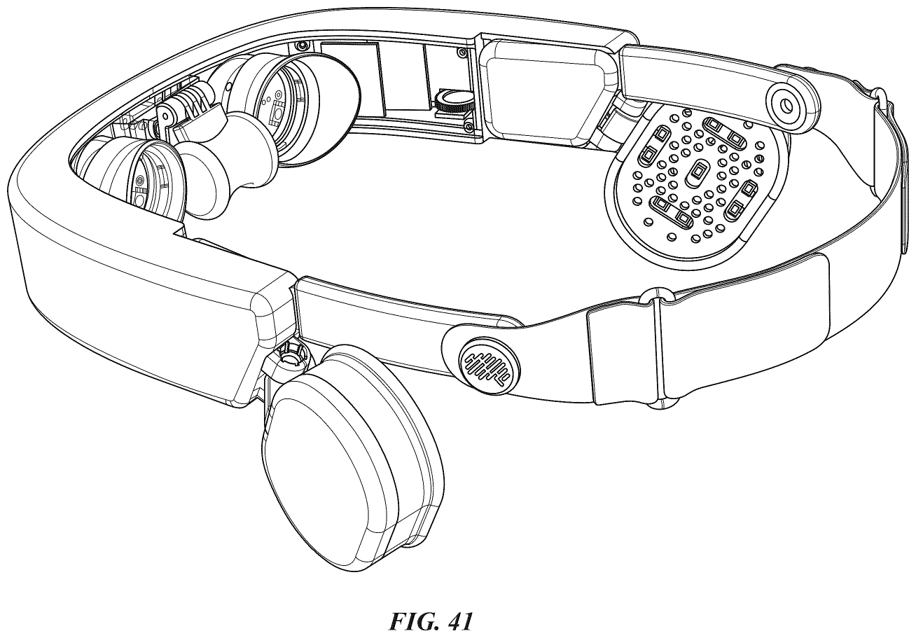

The helmet may deliver transcranial photobiomodulation. While not explicitly shown in FIG. 40 or 41, the helmet component may incorporate additional light-emitting elements designed to target specific regions of the brain.

FIG. 41 provides a rear view of the neurostimulation device, showing the curved headband structure that wraps around the circumference of the head. This view reveals the integration of the visor, headband (or headset), and audio components into a unified wearable form factor. The device may feature a modular construction that allows for the combination of multiple stimulation modalities.

In some cases, the audio and visual stimuli are precisely timed and synchronized to induce desired brainwave states. The neurostimulation system may generate specific brainwave patterns associated with relaxation, creativity, or deep sleep. By delivering carefully calibrated stimuli, the system may aim to modulate brainwave activity in a non-invasive manner.

The combination of auditory and visual entrainment provided by the headset and visor, along with the photobiomodulation capabilities of the helmet, may offer a comprehensive approach to neurostimulation. This integrated design may allow for simultaneous delivery of multiple forms of stimulation to potentially enhance the overall effectiveness of the therapy.

In some cases, the neurostimulation system may include a headset component designed to deliver audio signals to a user. As shown in FIG. 40, the headset may comprise headphone components attached to the sides of a curved headband structure. The headphones may feature circular ear cups connected to the main structure through adjustable mounting points, allowing for customized fit and comfort.

The headset component may be configured to generate and deliver specific audio signals, including binaural beats and isochronic tones. Binaural beats may be created when two slightly different frequencies are presented separately to each ear, resulting in the perception of a third tone at the frequency difference between the two original tones. Isochronic tones may involve a single tone that is turned on and off rapidly at regular intervals.

In some cases, the headset may be capable of producing audio signals within a frequency range of 18 to 0.5 Hz. This frequency range may be significant as it encompasses various brainwave states associated with different levels of consciousness and cognitive function. For example, frequencies near 18 Hz may correspond to beta waves associated with active, alert states, while frequencies approaching 0.5 Hz may align with delta waves typically observed during deep sleep or meditative states.

The headset component may utilize advanced audio processing techniques to generate precise binaural beats and isochronic tones within the specified frequency range. In some cases, the headset may incorporate digital signal processing capabilities to ensure accurate frequency production and timing of the audio signals.

As illustrated in FIG. 41, the headset component may be integrated into a larger neurostimulation device that includes a visor portion. The rear view shown in FIG. 41 demonstrates how the headphone components may be positioned in relation to the overall device structure, allowing for simultaneous delivery of audio and visual stimuli.

In some cases, the headset component may be designed to work in conjunction with other elements of the neurostimulation system, such as the visor and helmet components. The integration of these components may allow for synchronized delivery of audio, visual, and photobiomodulation stimuli to potentially enhance the overall effectiveness of the neurostimulation therapy.

The ability of the headset to deliver audio signals within the 18 to 0.5 Hz range may provide flexibility in targeting specific brainwave states. For instance, audio signals in the higher end of this range may be used to promote alertness or focus, while lower frequencies may be employed to induce relaxation or sleep. This versatility may allow the neurostimulation system to be adapted for various therapeutic applications or cognitive enhancement purposes.

In some cases, the neurostimulation system may include a visor component designed to deliver visual signals to a user. As shown in FIG. 40, the visor portion extends across the front of the headband structure and is designed to cover the user's eyes. The visor may incorporate circular housings integrated into the headband structure, which may contain optical components for delivering visual stimulation.

The visor component may be configured to emit pulsed light at specific frequencies and wavelengths. In some cases, the visor may utilize light-emitting diodes (LEDs) that emit light at a wavelength of 470 nanometers. This particular wavelength falls within the blue light spectrum and may be chosen for its potential effects on brain activity and circadian rhythms.

The LEDs in the visor may be designed to flicker at frequencies ranging from 18 Hz to 0.5 Hz. This frequency range encompasses various brainwave states associated with different levels of consciousness and cognitive function. For example, frequencies near 18 Hz may correspond to beta waves associated with active, alert states, while frequencies approaching 0.5 Hz may align with delta waves typically observed during deep sleep or meditative states.

In some cases, the flickering light emitted by the visor may be used to induce specific brainwave patterns through a process known as visual entrainment. This technique may involve synchronizing the brain's electrical activity with the frequency of the flickering light, potentially influencing the user's mental state or cognitive performance.

As illustrated in FIG. 41, the visor component may be integrated into the overall structure of the neurostimulation device. The rear view shown in FIG. 41 demonstrates how the visor portion wraps around the front and sides of the head, allowing for comprehensive visual stimulation coverage.

The visual signals delivered by the visor may work in conjunction with other elements of the neurostimulation system, such as the headset and helmet components. In some cases, the combination of visual stimulation from the visor and audio signals from the headset may provide a more potent effect than visual or audio stimulation alone.

The ability of the visor to deliver light at 470 nanometers and flicker within the 18 to 0.5 Hz range may offer flexibility in targeting specific brainwave states. For instance, higher flickering frequencies may be used to promote alertness or focus, while lower frequencies may be employed to induce relaxation or sleep. This versatility may allow the neurostimulation system to be adapted for various therapeutic applications or cognitive enhancement purposes. In some cases, the neurostimulation system may include a helmet component designed to deliver transcranial photobiomodulation to a user. While not explicitly shown in FIG. 40 or 41, the helmet component may be integrated with the headset and visor components to provide a comprehensive neurostimulation device.

More specifically a detailed description on a figure-by-figure basis is included herein as follows;

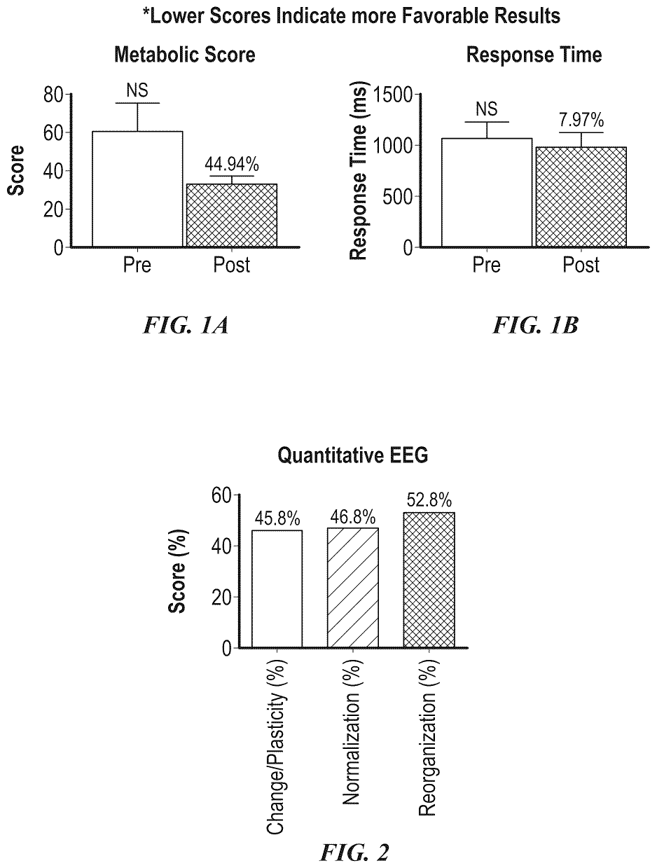

FIGS. 1A and 1B illustrate a set of bar graphs showing the effects of Audiovisual Brain Entrainment therapy on metabolic score and response time. In FIG. 1A, the graph displays a comparison of metabolic scores between pre-treatment and post-treatment measurements. The pre-treatment score is shown to be higher than the post-treatment score, with a 44.94% reduction observed after treatment. FIG. 1B presents response time measurements between pre-treatment and post-treatment conditions. The post-treatment response time demonstrates a 7.97% reduction compared to pre-treatment. Both graphs include error bars to indicate the variability of the measurements. The changes in both metabolic score and response time are marked with “NS,” indicating that the observed differences were not statistically significant. (Paired T-Test 95% confidence interval)

FIG. 2 depicts a bar graph illustrating quantitative EEG analysis results from audiovisual brain entrainment therapy. The graph displays three distinct measurements: Change/Plasticity at 45.8%, Normalization at 46.8%, and Reorganization at 52.8%. The vertical axis represents Score values as percentages ranging from 0 to 60, while the horizontal axis lists the three measured parameters. To visually distinguish between the three measurements, the bars are differentiated using white, hatching, and cross-hatching fill patterns. This representation allows for a clear comparison of the different aspects of brain activity changes resulting from the therapy.

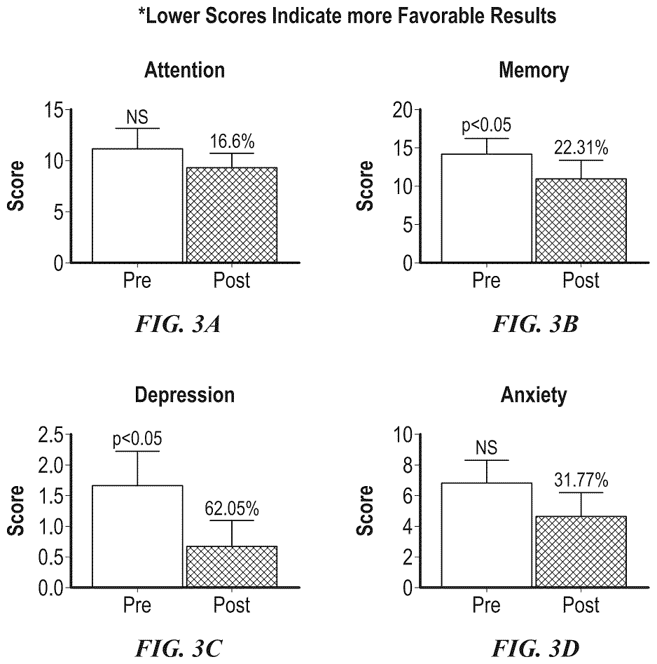

FIGS. 3A through 3D show a set of bar graphs illustrating the effects of Audiovisual Brain Entrainment therapy on cognitive and emotional measures, displaying results for Attention, Memory, Depression, and Anxiety scores respectively. Each panel compares Pre and Post intervention measurements, with white bars representing Pre scores and cross-hatched bars representing Post scores. FIG. 3A shows Attention scores with a non-significant decrease of 16.6% from Pre to Post intervention. FIG. 3B demonstrates Memory scores with a statistically significant decrease of 22.31% (p<0.05) from Pre to Post intervention. FIG. 3C illustrates Depression scores with a statistically significant reduction of 62.05% (p<0.05) from Pre to Post intervention. FIG. 3D displays Anxiety scores with a non-significant decrease of 31.77% from Pre to Post intervention. Error bars are included on all measurements to indicate variability. A note at the bottom of the figure indicates that lower scores represent more favorable results across all measures. (Paired T-Test 95% confidence interval)

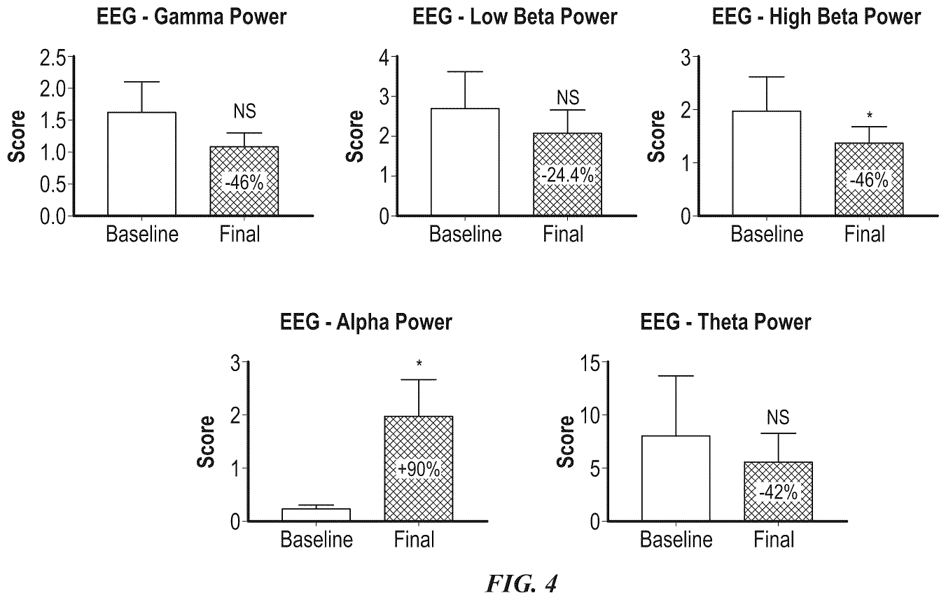

FIG. 4 presents a set of bar graphs showing EEG power measurements across different brainwave frequencies in college golf players. The graphs compare baseline and final measurements for five different brainwave frequency bands: Gamma Power, Low Beta Power, High Beta Power, Alpha Power, and Theta Power. Each graph displays the baseline measurement in white and the final measurement in cross-hatching, with percentage changes indicated. The Gamma Power graph shows a 46% decrease from baseline to final measurement. Low Beta Power exhibits a 24.4% decrease. High Beta Power demonstrates a statistically significant 46% decrease, marked with an asterisk. Alpha Power shows a statistically significant 90% increase, also marked with an asterisk. The Theta Power graph indicates a 42% decrease from baseline to final measurement. Error bars are included on each measurement to indicate the range of variation in the data. (Paired T-Test 95% confidence interval)

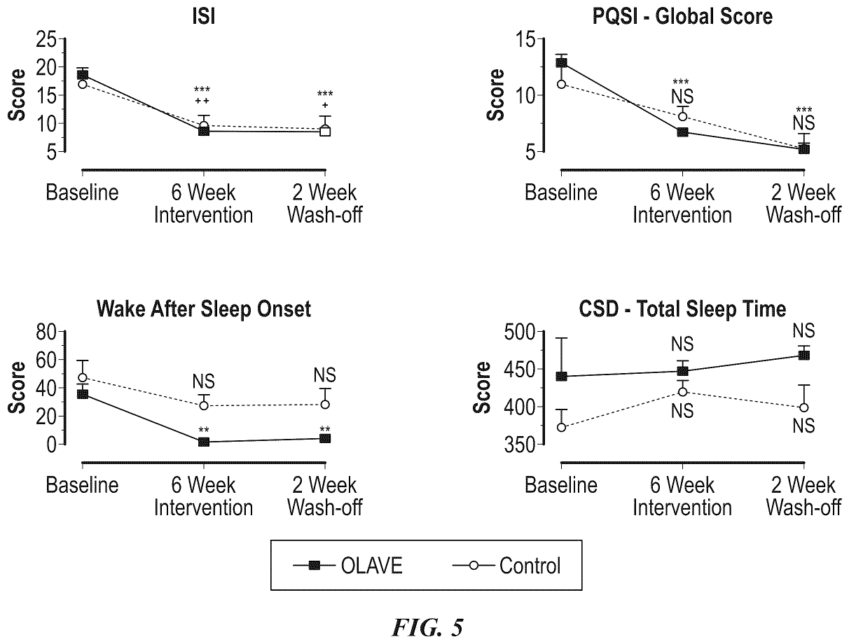

FIG. 5 illustrates four graphs showing the effects of open-loop audio-visual entrainment (OLAVE) on sleep parameters compared to a control group. The top left graph displays Insomnia Severity Index (ISI) scores decreasing over time for both OLAVE and control groups across baseline, 6-week intervention, and 2-week wash-off periods. The top right graph shows Pittsburgh Quality of Sleep Index (PQSI) Global Scores declining more substantially in the OLAVE group compared to control across the same time periods. The bottom left graph illustrates Wake After Sleep Onset (WASO) measurements decreasing more notably in the OLAVE group versus control from baseline through the intervention and wash-off periods. The bottom right graph displays Consensus Sleep Diary (CSD) Total Sleep Time increasing in the OLAVE group while remaining relatively stable in the control group across the measurement periods. (Two-way ANOVA repeated measures followed by Bonferroni Post-hoc Test)

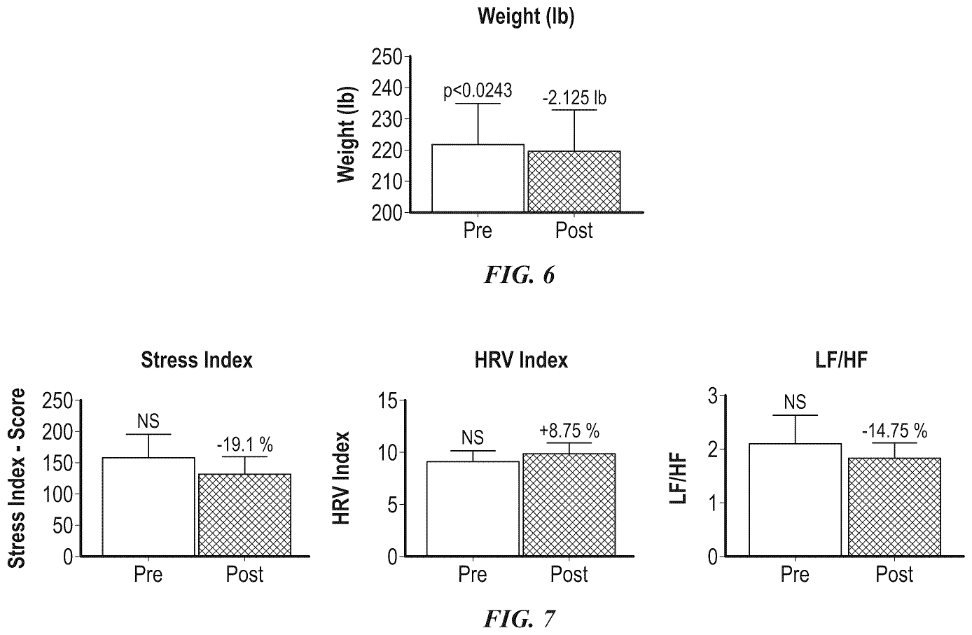

FIG. 6 and FIG. 7 depict bar graphs showing the effects of audiovisual brainwave entrainment on physiological measurements. FIG. 6 displays weight measurements before and after treatment, with a statistically significant decrease of 2.125 pounds (p<0.0243) from pre-treatment to post-treatment conditions (Paired T-Test 95% confidence interval). FIG. 7 shows three physiological measurements: stress index demonstrating a 19.1% reduction, HRV index showing an 8.75% increase, and LF/HF ratio displaying a 14.75% decrease from pre-treatment to post-treatment conditions. These changes in the lower graph are marked with “NS” labels, indicating that they were not statistically significant (Paired T-Test 95% confidence interval).

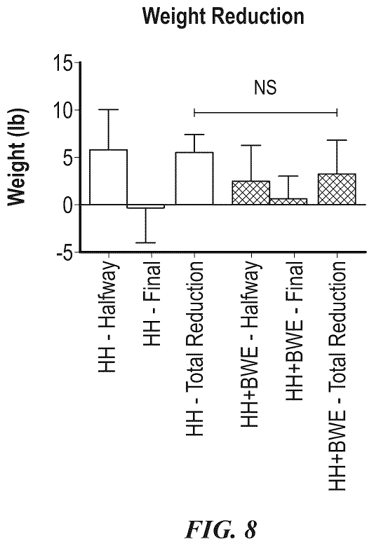

FIG. 8 illustrates a bar graph showing weight reduction results from a 13-week program comparing two treatment conditions. The graph displays weight measurements in pounds on the y-axis, with data points shown for halfway and final measurements, as well as total reduction values. The first condition shows results for the H.E.A.L.T.H.Y. H.A.B.I.T.S. (HH) program alone, represented by white bars, while the second condition shows results for the program combined with Brainwave Entrainment, represented by cross-hatched bars. A horizontal line labeled “NS” indicates that the difference between the two conditions was not statistically significant. (Unpaired T-Test 95% confidence interval)

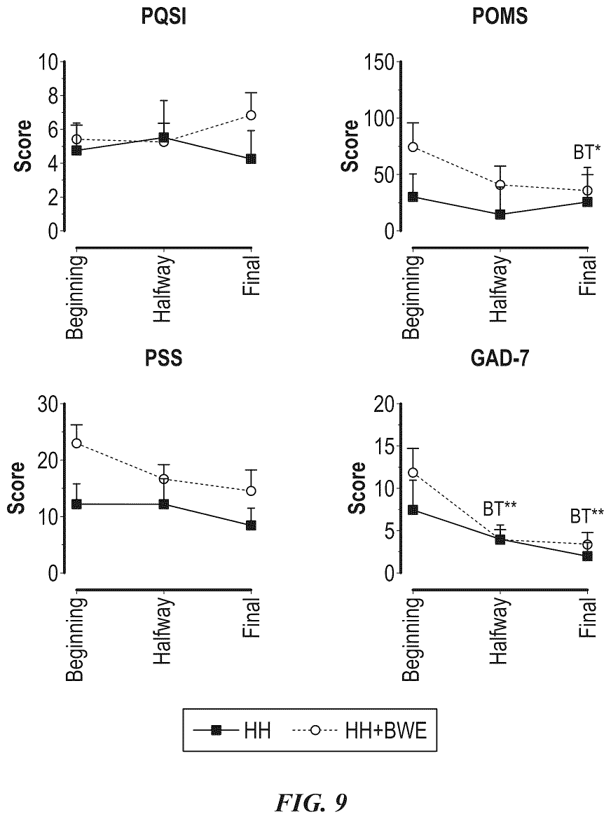

FIG. 9 presents four graphs showing the effects of a 13-week health program on various psychological and physiological measures. The graphs compare two treatment conditions: HH (program alone) shown with black squares and HH+BWE (program with Brainwave Entrainment) shown with gray circles, measured at three timepoints: Beginning, Halfway, and Final. The top left graph shows Pittsburgh Quality of Sleep Index (PQSI) scores, with both groups starting at similar baseline values around 5 points. The top right graph displays Profile of Mood States (POMS) scores, with the HH+BWE group showing higher initial values around 75 points compared to the HH group at approximately 25 points. The bottom left graph illustrates Perceived Stress Scale (PSS) scores, with the HH+BWE group demonstrating higher baseline values around 22 points versus the HH group at approximately 12 points. The bottom right graph shows Generalized Anxiety Disorder-7 (GAD-7) scores, with both groups showing decreasing trends over time and statistical significance indicated by “BT” markers. (Two-way ANOVA repeated measures followed by Bonferroni Post-hoc Test)

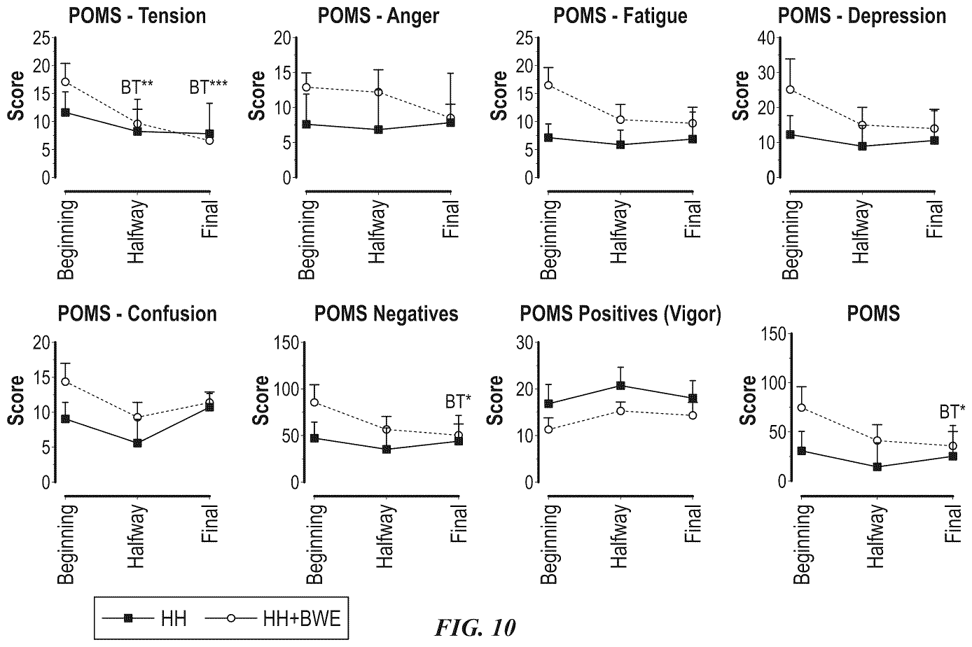

FIG. 10 depicts a set of line graphs showing the effects of a 13-week program on various Profile of Mood States (POMS) subscales. The graphs compare two treatment conditions—HH (program alone) and HH+BWE (program plus Brainwave Entrainment)—measured at beginning, halfway, and final timepoints. The graphs display results for eight different POMS measures: tension, anger, fatigue, depression, confusion, POMS negatives, POMS positives (vigor), and total POMS score. The tension subscale shows a significant decrease marked as “BT” and “BT” for the HH+BWE condition compared to HH. The total POMS score shows a significant difference marked as “BT” between the conditions. The graphs include error bars indicating measurement variability and statistical significance markers where differences between conditions were found. (Two-way ANOVA repeated measures followed by Bonferroni Post-hoc Test)

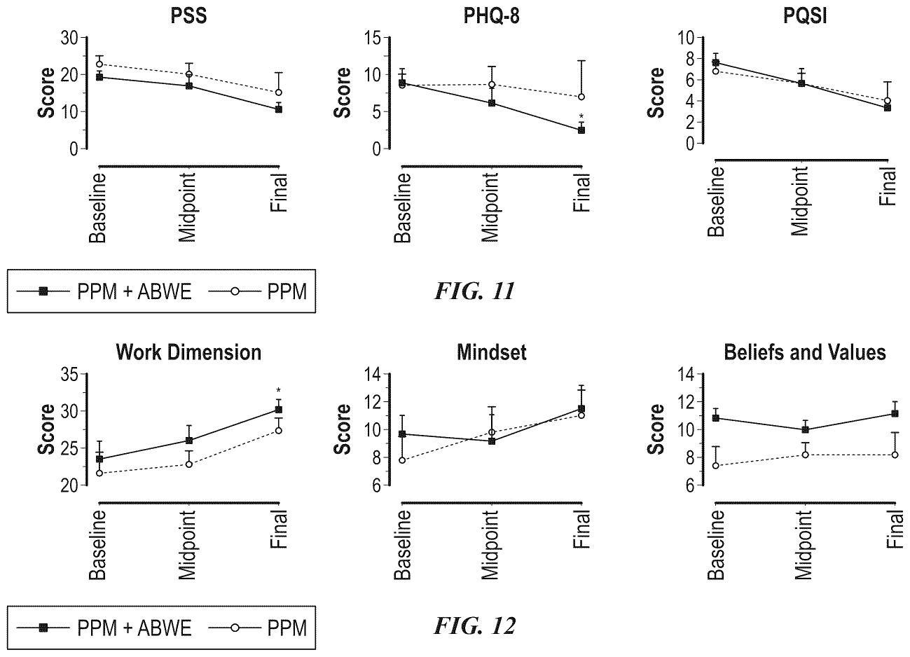

FIG. 11 and FIG. 12 illustrate line graphs showing the effects of Peak Performance Method (PPM) alone and in combination with Audio Brainwave Entrainment (ABWE) across different assessment dimensions. In FIG. 11, The graphs display scores for PPM alone and PPM+ABWE on Perceived Stress Scale (PSS), Patient Health Questionnaire (PHQ-8), and Pittsburgh Quality of Sleep Index (PQSI). FIG. 12 displays scores for Work Dimension, Mindset, and Beliefs and Values measured at baseline, midpoint, and final timepoints for two treatment groups—PPM alone and PPM+ABWE. The Work Dimension graph shows an increasing trend in scores over time for both groups, with the PPM+ABWE group achieving higher final scores compared to PPM alone. The Mindset graph demonstrates relatively stable scores from baseline to midpoint followed by slight increases at the final timepoint for both groups. The Beliefs and Values graph indicates minimal changes across measurement periods, with the PPM alone group showing a slight increase while the PPM+ABWE group maintains more stable scores throughout the study period. (Two-way ANOVA repeated measures followed by Bonferroni Post-hoc Test)

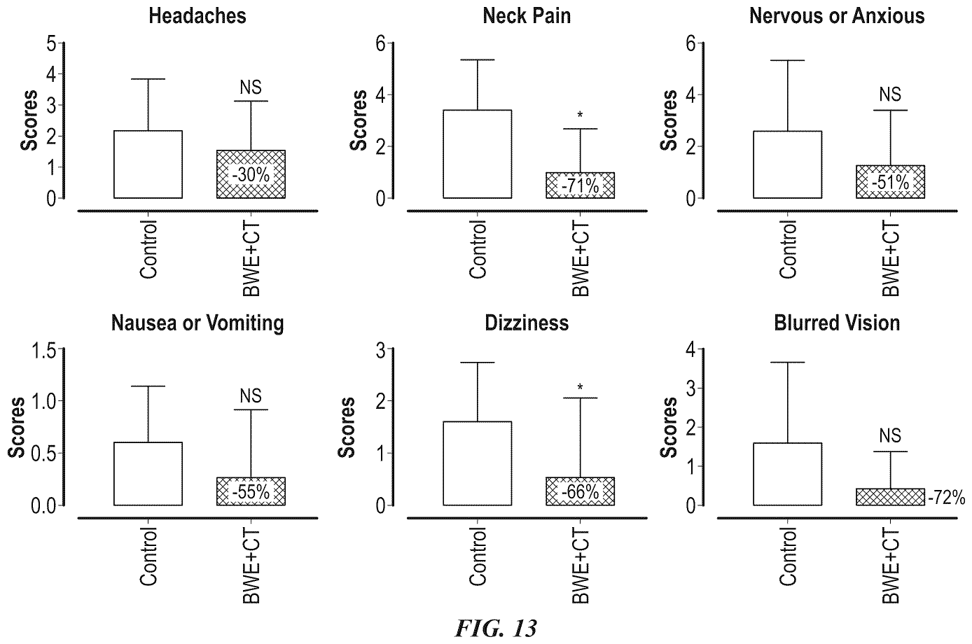

FIG. 13 shows a bar graph comparing symptom scores between a control group and a BWE+CT (Brainwave Entrainment with Compression Therapy) treatment group. The graph displays six different symptoms: headaches, neck pain, nervous or anxious feelings, nausea or vomiting, dizziness, and blurred vision. The BWE+CT group demonstrates percentage reductions in symptoms ranging from 30% to 72% compared to the control group. Neck pain and dizziness show statistically significant reductions of 71% and 66% respectively, as indicated by asterisks. The remaining symptoms show non-statistically significant reductions, with headaches reduced by 30%, nervous/anxious symptoms reduced by 51%, nausea/vomiting reduced by 55%, and blurred vision reduced by 72%. (Paired T-Test 95% confidence interval)

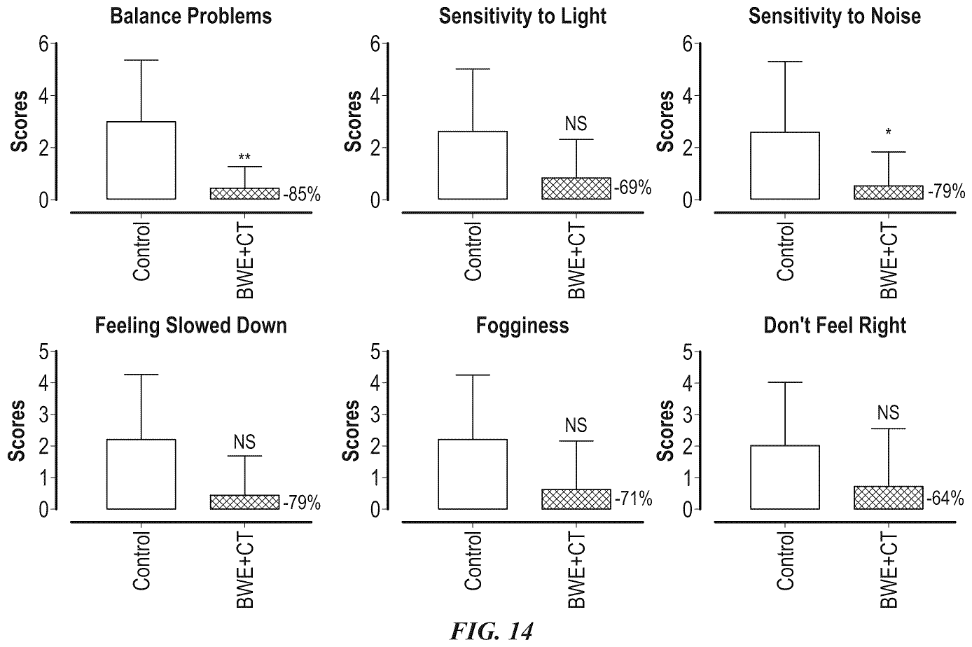

FIG. 14 presents a set of bar graphs comparing concussion-related symptoms between a control group and a BWE-CT (Brainwave Entrainment with Compression Therapy) treatment group. The graphs show measurements for six different symptoms: balance problems, sensitivity to light, sensitivity to noise, feeling slowed down, fogginess, and “don't feel right.” The control group measurements are represented by white bars while the BWE-CT group measurements are shown in cross-hatched bars. The graphs indicate percentage reductions in symptoms ranging from 64% to 85% for the BWE-CT group compared to the control group, with balance problems showing a statistically significant reduction of 85% and sensitivity to noise showing a statistically significant reduction of 79%. The remaining symptoms of sensitivity to light, feeling slowed down, fogginess, and “don't feel right” show varying degrees of reduction in the BWE-CT group compared to the control group, though these changes are marked as not statistically significant. (Paired T-Test 95% confidence interval).

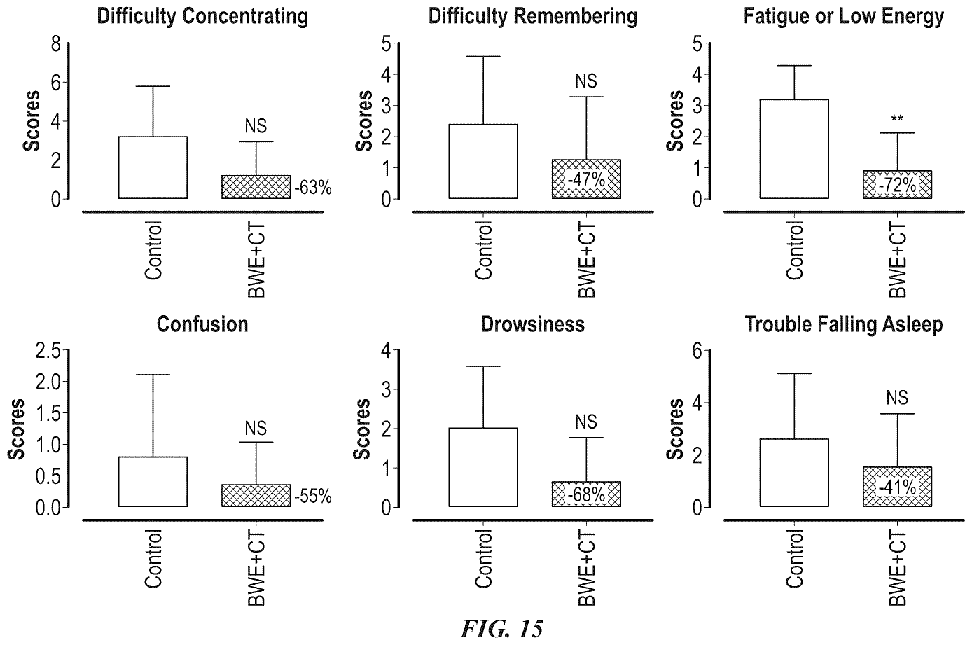

FIG. 15 illustrates a set of bar graphs comparing concussion-related symptoms between a control group and a BWE+CT (Brainwave Entrainment with Compression Therapy) treatment group. The graphs show measurements for six different symptoms: difficulty concentrating, difficulty remembering, fatigue or low energy, confusion, drowsiness, and trouble falling asleep. Each symptom displays two bars-a white bar representing the control group and a cross-hatched bar representing the BWE+CT group. The BWE+CT group shows percentage reductions ranging from 41% to 72% compared to the control group across the different symptoms. The reduction in fatigue or low energy shows statistical significance as indicated by double asterisks, while other symptoms are marked as not statistically significant (NS). (Paired T-Test 95% confidence interval).

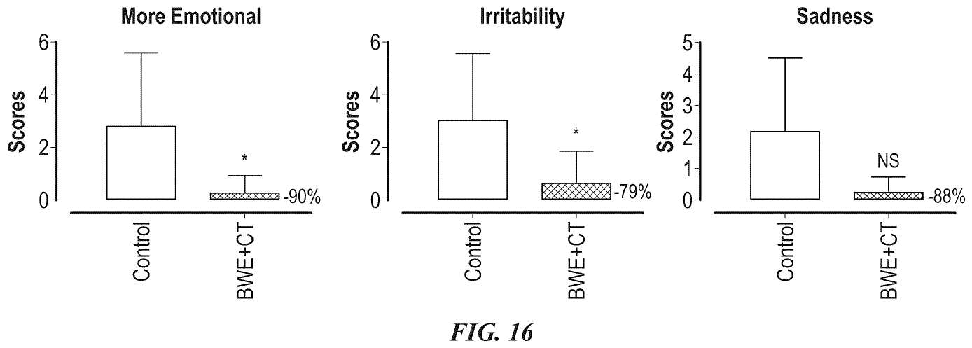

FIG. 16 depicts three bar graphs comparing emotional symptoms between a control group and a BWE-CT (Brainwave Entrainment with Compression Therapy) treatment group. The first graph shows “More Emotional” scores, with the BWE-CT group demonstrating a 90% reduction compared to the control group, marked with an asterisk indicating statistical significance (p<0.05). The second graph displays “Irritability” scores, with the BWE-CT group showing a 79% reduction compared to the control group, also marked with an asterisk indicating statistical significance (p<0.05). The third graph illustrates “Sadness” scores, with the BWE-CT group showing an 88% reduction compared to the control group, marked with “NS” indicating the difference was not statistically significant. (Paired T-Test 95% confidence interval).

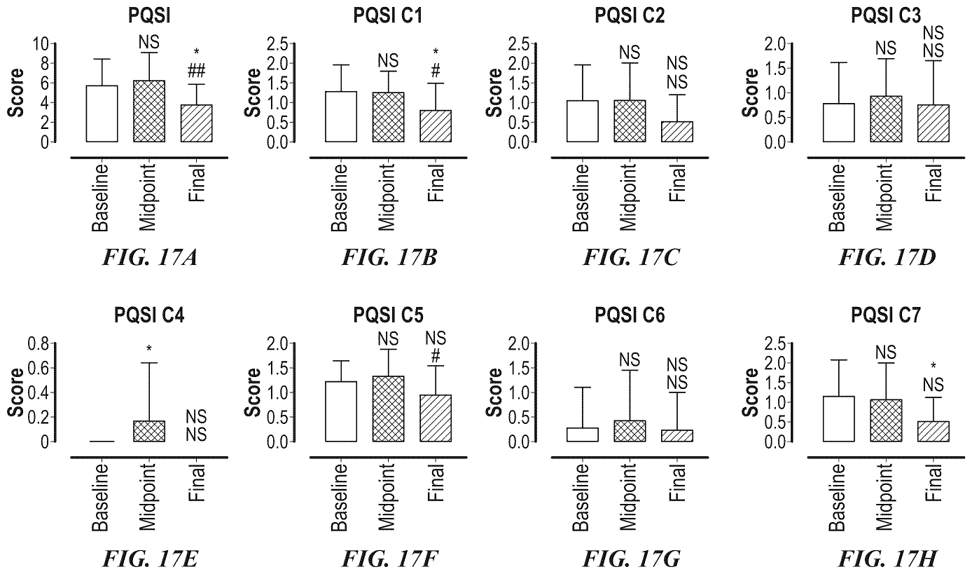

FIGS. 17A through 17H show a bar graph illustrating Pittsburgh Quality of Sleep Index (PQSI) scores measured at three different time points: baseline, midpoint, and final evaluation. The graphs of FIG. 17A through FIG. 17H, each show different sleep-related components. FIG. 17A shows the global PQSI score, while FIG. 17B through FIG. 17H display individual component scores including subjective sleep quality, sleep latency, sleep duration, sleep efficiency, sleep disturbance, use of sleep medication, and daytime dysfunction. The bars in each panel represent mean values with error bars indicating standard deviation, and statistical significance is denoted by asterisks and “NS” (not statistically significant) labels above the bars. (Ordinary One-Way ANOVA followed by Tukey's multiple comparisons test or Kruskal-Wallis test followed by Dunn's multiple comparison test, where applicable)

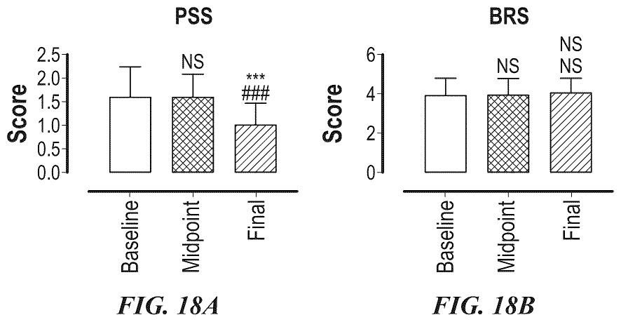

FIGS. 18A and 18B present two bar graphs showing changes in psychological measures over time. FIG. 18A shows the Perceived Stress Scale (PSS) scores measured at baseline, midpoint, and final timepoints. The PSS scores remain relatively stable between baseline and midpoint measurements, followed by a significant decrease at the final timepoint. FIG. 18B shows the Brief Resilience Scale (BRS) scores measured at the same three timepoints. The BRS scores demonstrate minimal variation across all three measurement periods, with no statistically significant changes observed between timepoints. (Ordinary One-Way ANOVA followed by Tukey's multiple comparisons test)

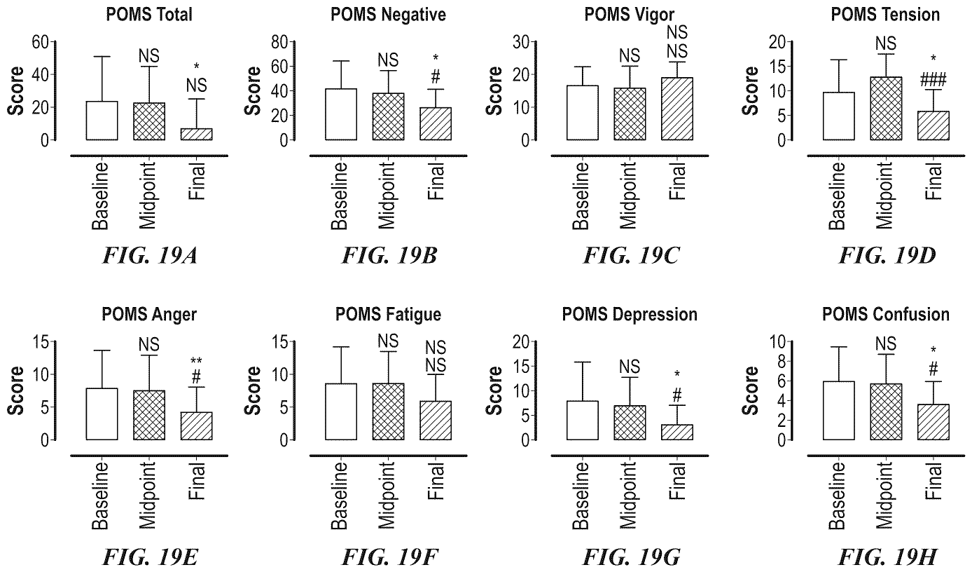

FIGS. 19A-19H provide graphs showing Profile of Mood States (POMS) measurements across multiple parameters. The graphs (FIGS. 19A-19H) display different mood state measurements including POMS total score, POMS negative aspects, POMS vigour, POMS tension, POMS anger, POMS fatigue, POMS depression, and POMS confusion. Each figure compares scores at baseline, midpoint, and final evaluation timepoints. The bars include error bars indicating statistical variance, with notations indicating statistical significance levels using asterisks and “NS” (not statistically significant) labels. The y-axis scales vary between panels to appropriately display the range of scores for each mood parameter, while the x-axis consistently shows the three timepoints of baseline, midpoint, and final measurements. (Ordinary One-Way ANOVA followed by Tukey's multiple comparisons test or Kruskal-Wallis test followed by Dunn's multiple comparison test, where applicable)

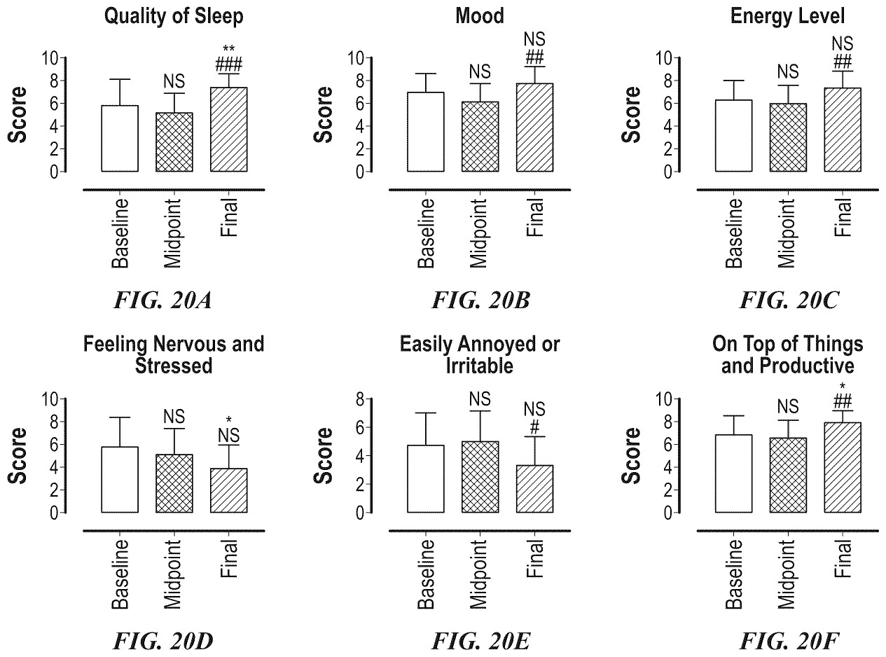

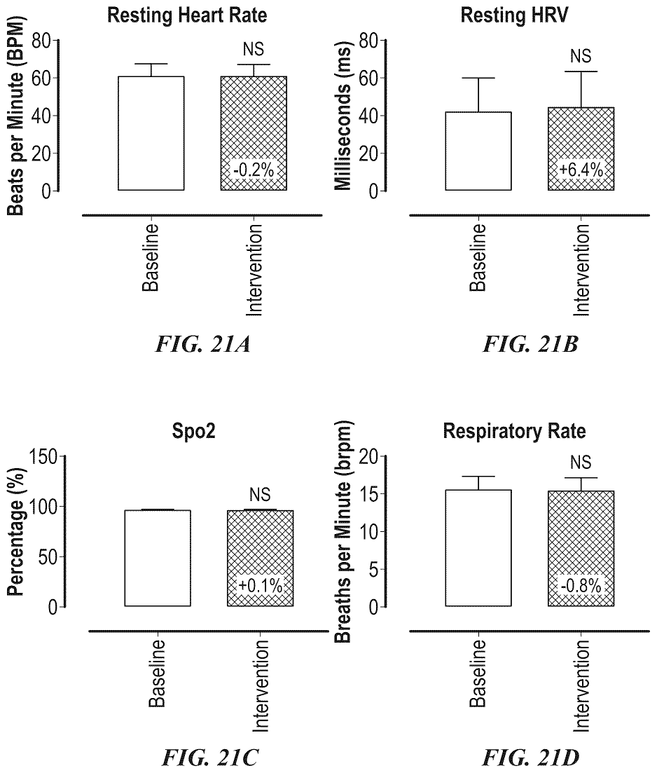

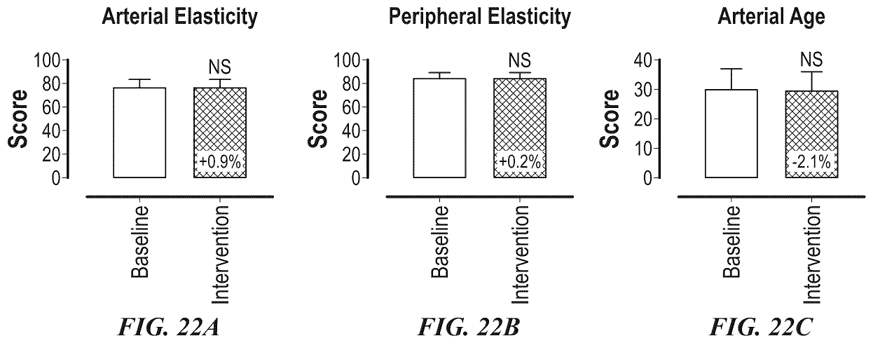

FIGS. 20A-20F depicts a set of bar graphs showing self-reported measurements on a 10-point Likert scale across multiple time points. The graphs display data for six different metrics: quality of sleep, mood, energy level, feeling nervous and stressed, feeling easily annoyed or irritable, and feeling “on top of things” and productive. Each graph shows measurements taken at baseline, midpoint, and final evaluation periods, with white bars representing baseline, cross-hatched bars representing midpoint, and hatched bars representing final measurements. Statistical significance indicators show where changes were significant compared to baseline (marked with asterisks) or midpoint (marked with pound signs) evaluations, while “NS” indicates changes that were not statistically significant. (Ordinary One-Way ANOVA followed by Tukey's multiple comparisons test or Kruskal-Wallis test followed by Dunn's multiple comparison test, where applicable)

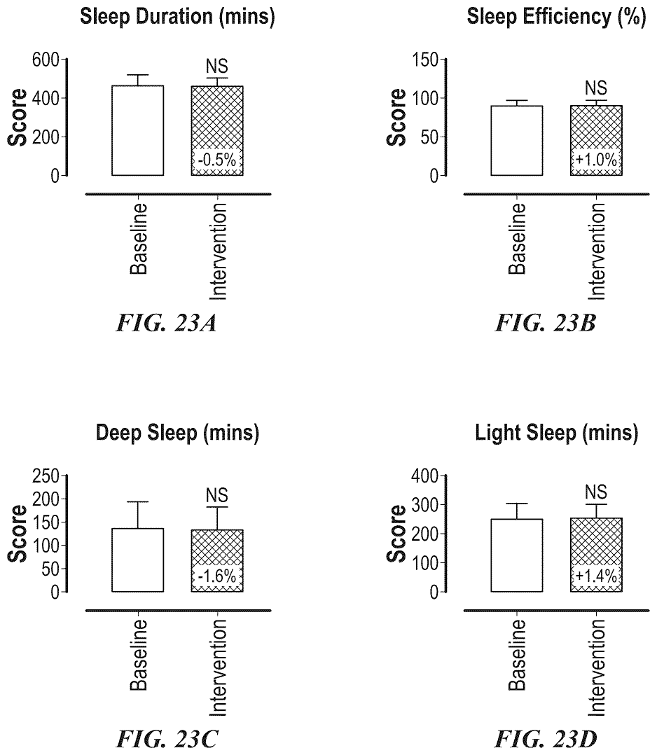

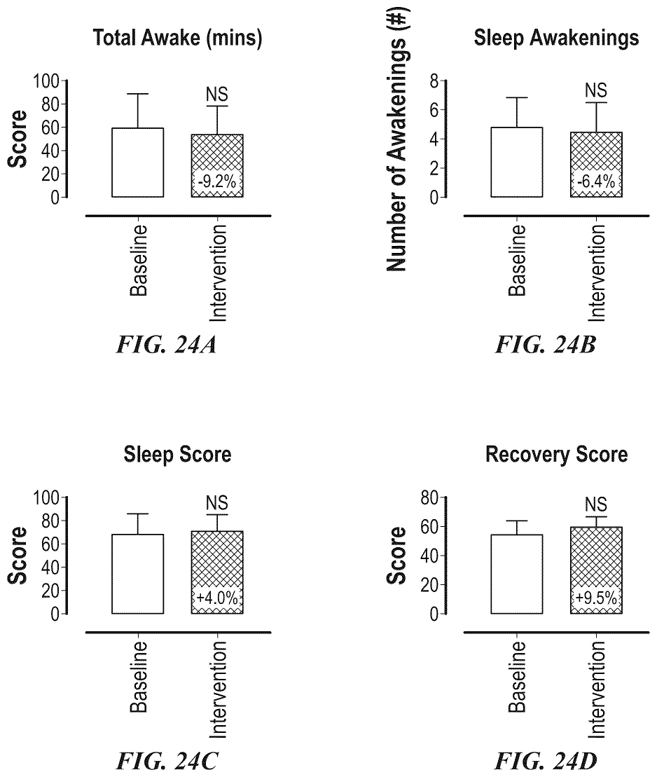

FIGS. 21A-21D show a set of bar graphs displaying physiological measurements before and after an intervention. The graphs present four different measurements: resting heart rate in beats per minute, resting heart rate variability (HRV) in milliseconds, peripheral capillary oxygen saturation (SpO2) as a percentage, and respiratory rate in breaths per minute. The baseline measurements are shown as white bars while the intervention measurements are shown as black bars. The resting heart rate shows a −0.2% change, the resting HRV shows a +6.4% change, the SpO2 shows a +0.1% change, and the respiratory rate shows a −0.8% change from baseline to intervention. Each graph includes error bars and is marked with “NS” indicating the changes were not statistically significant. (Unpaired or paired T-Test or Wilcoxon matched-pairs signed rank test, where applicable)

FIGS. 22A-22C illustrate three bar graphs comparing baseline and intervention measurements for arterial elasticity, peripheral elasticity, and arterial age. FIG. 22A shows arterial elasticity scores with a baseline measurement and an intervention measurement that indicates a 0.9% increase, though this change is noted as not statistically significant (NS). FIG. 22B displays peripheral elasticity scores with baseline and intervention measurements showing a 0.2% increase, also marked as not statistically significant. FIG. 22C presents arterial age measurements comparing baseline and intervention values, showing a 2.1% decrease that is indicated as not statistically significant. (Unpaired or paired T-Test or Wilcoxon matched-pairs signed rank test, where applicable)

FIGS. 23A-23D depicts a set of bar graphs comparing sleep-related measurements between baseline and intervention conditions. The graphs show four different sleep parameters measured at baseline and after intervention, with error bars indicating variance. FIG. 23A displays sleep duration in minutes, showing a slight decrease of 0.5% from baseline to intervention that is not statistically significant. FIG. 23B illustrates sleep efficiency as a percentage, demonstrating a small increase of 1.0% from baseline to intervention that is not statistically significant. FIG. 23C shows deep sleep duration in minutes, indicating a decrease of 1.6% from baseline to intervention that is not statistically significant. FIG. 23D presents light sleep duration in minutes, showing an increase of 1.4% from baseline to intervention that is not statistically significant. (Unpaired or paired T-Test or Wilcoxon matched-pairs signed rank test, where applicable)

FIGS. 24A-24D present a set of bar graphs comparing sleep-related measurements between baseline and intervention conditions. The graphs show four different sleep parameters measured at baseline and after intervention, with error bars indicating variance. Panel A displays Total Awake time in minutes, showing a 9.2% decrease from baseline to intervention. Panel B illustrates Sleep Awakenings frequency, demonstrating a 6.4% reduction from baseline to intervention. Panel C presents Sleep Score data, indicating a 4.0% increase from baseline to intervention. Panel D shows Recovery Score measurements, revealing a 9.5% improvement from baseline to intervention. The graphs indicate that none of the changes between baseline and intervention reached statistical significance, as denoted by “NS” labels. (Unpaired or paired T-Test or Wilcoxon matched-pairs signed rank test, where applicable)

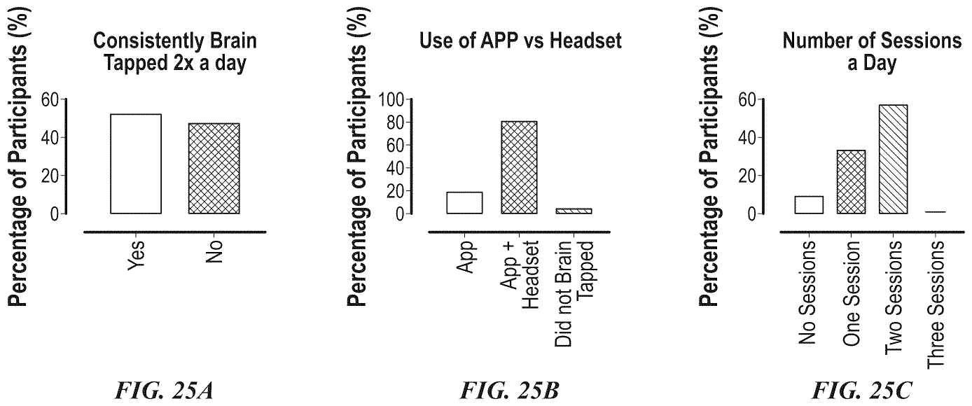

FIGS. 25A-25C illustrate three bar graphs showing adherence data for a BrainTapping intervention study. FIG. 25A shows the percentage of participants who consistently BrainTapped twice per day during the intervention phase, with approximately 50% responding “Yes” and 50% responding “No”. FIG. 25B compares the use of different BrainTapping methods, showing that approximately 80% of participants used a headset, while smaller percentages used either the app alone or did not BrainTap. FIG. 25C displays the number of BrainTapping sessions performed per day, with the majority of participants completing two sessions daily, followed by one session, and smaller percentages doing either no sessions or three sessions.

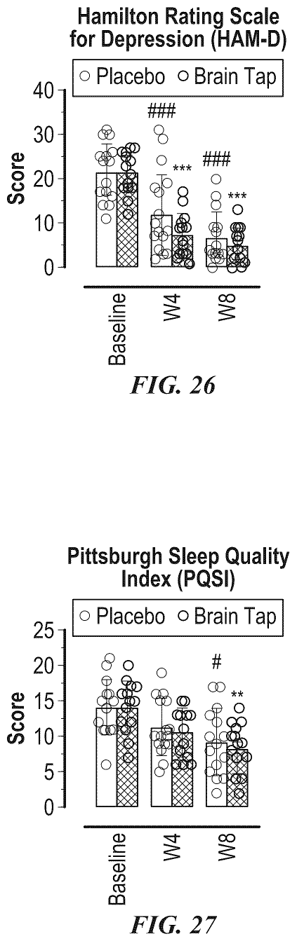

FIG. 26 evaluates symptoms of depression using the Hamilton rating Scale for Depression (HAM-D) between placebo and BrainTap groups over time. The graphs compare results between a placebo group and a BrainTap treatment group at three timepoints: baseline, week 4 (W4), and week 8 (W8). The HAM-D graph indicates decreasing depression levels across both groups over the 8-week period, with the BrainTap group showing lower depression scores compared to placebo at the final timepoint.

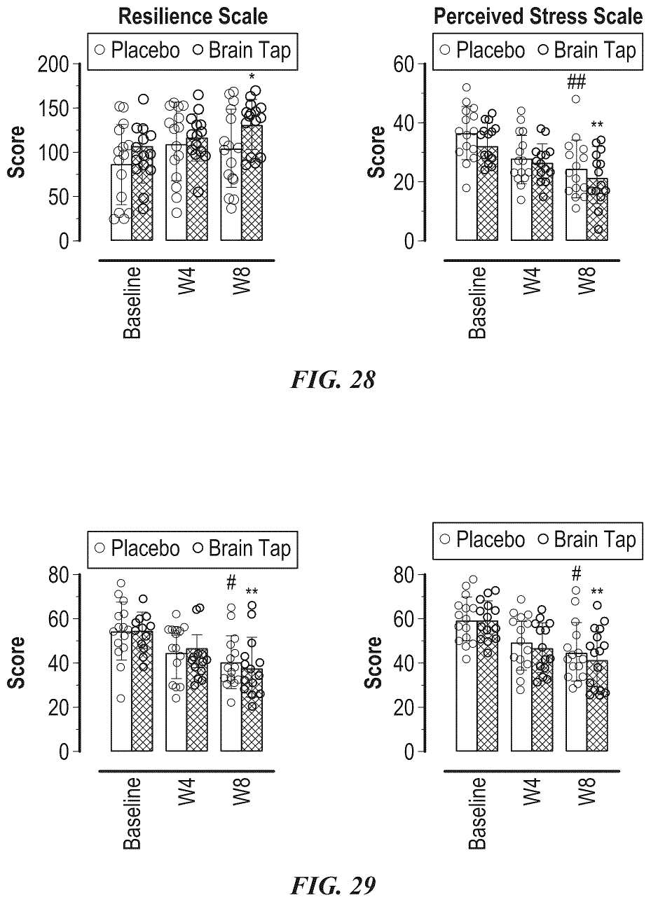

FIG. 27 and FIG. 28 depict three bar graphs showing sleep quality, resilience, and perceived stress measurements over time. The graphs compare results between a placebo group and a BrainTap treatment group at three timepoints: baseline, week 4 (W4), and week 8 (W8). The Pittsburgh Sleep Quality Index graph (FIG. 27) shows sleep quality scores decreasing over the 8-week period for both groups, with the BrainTap group showing a more pronounced reduction by week 8. The Resilience Scale graph of FIG. 28 displays increasing resilience scores over time, with the BrainTap group demonstrating higher scores compared to placebo by week 8. The Perceived Stress Scale graph of FIG. 28 indicates decreasing stress levels across both groups over the 8-week period, with the BrainTap group showing lower stress scores compared to placebo at the final timepoint.

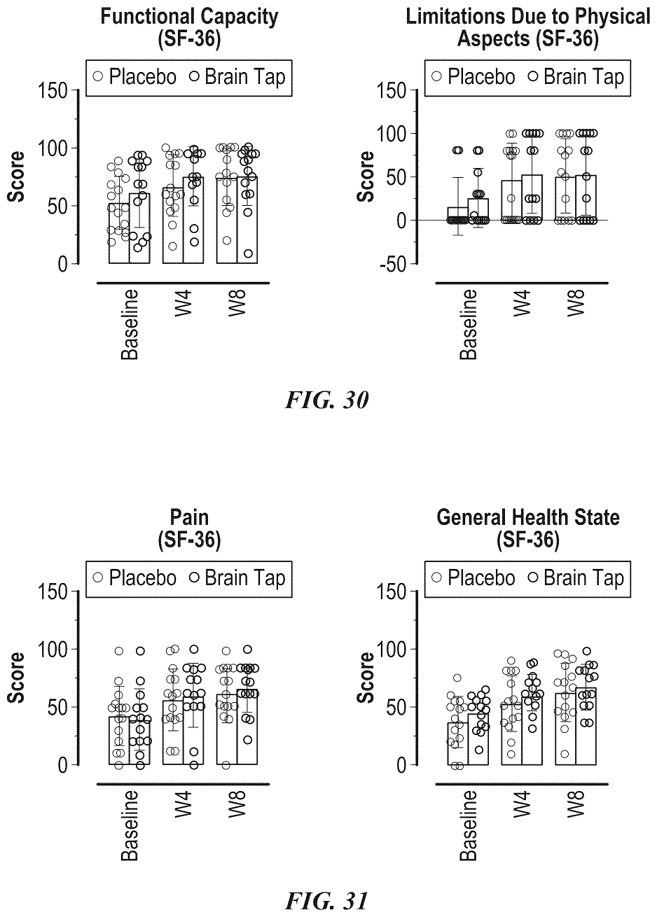

FIG. 29 and FIG. 30 show two sets of bar graphs comparing anxiety scores between placebo and Brain Tap treatment groups over time. The graphs of FIG. 29 display State-Trait Anxiety Inventory (STAI) scores measured at baseline, week 4 (W4), and week 8 (W8) timepoints. The left graph displays individual data points overlaid on bars representing group means, while the right graph shows similar data with a different distribution pattern. The graphs of FIG. 30 display Functional Capacity and Limitations due to Physical Aspects scores measured at baseline, week 4 (W4), and week 8 (W8) timepoints. Both graphs use white bars to represent the placebo group and cross-hatched bars to represent the BrainTap treatment group, with error bars indicating score variability within each group.

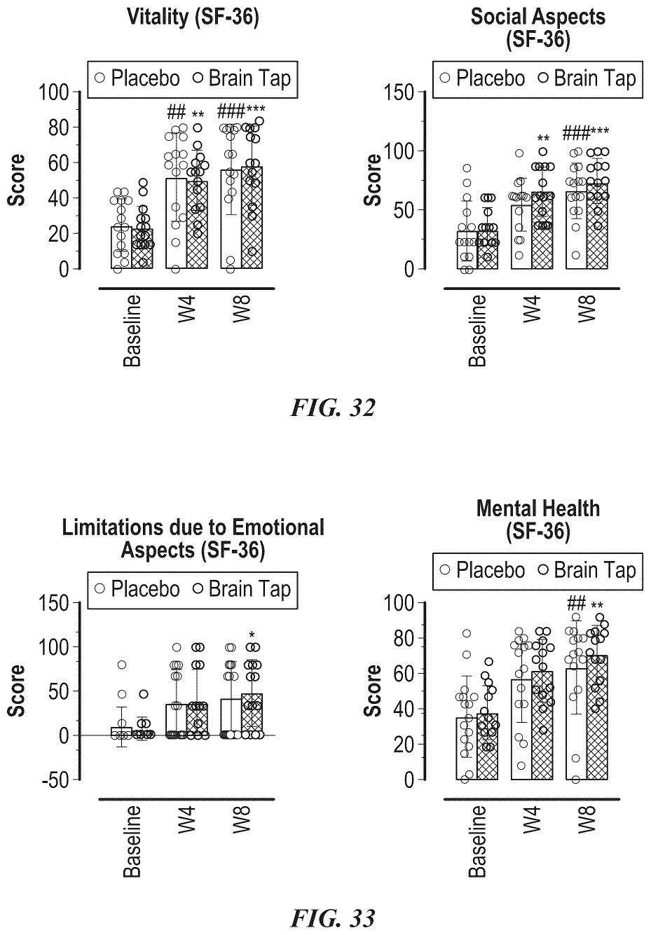

FIG. 31 and FIG. 32 illustrate graphs showing results from the Medical Outcomes Short-Form Health Survey (SF-36) comparing placebo and BrainTap treatments. The graphs of FIG. 31 display scores for pain and general health state, while FIG. 32 displays vitality, and social aspects measured at baseline, week 4 (W4), and week 8 (W8). The pain scores show similar trends between placebo and BrainTap groups across the measurement periods. The general health state scores demonstrate comparable patterns between the two groups over time. The vitality scores indicate differences between placebo and BrainTap groups, with notable increases from baseline to W4 and W8 timepoints. The social aspects scores show distinctions between the groups, with changes observed across the measurement periods.

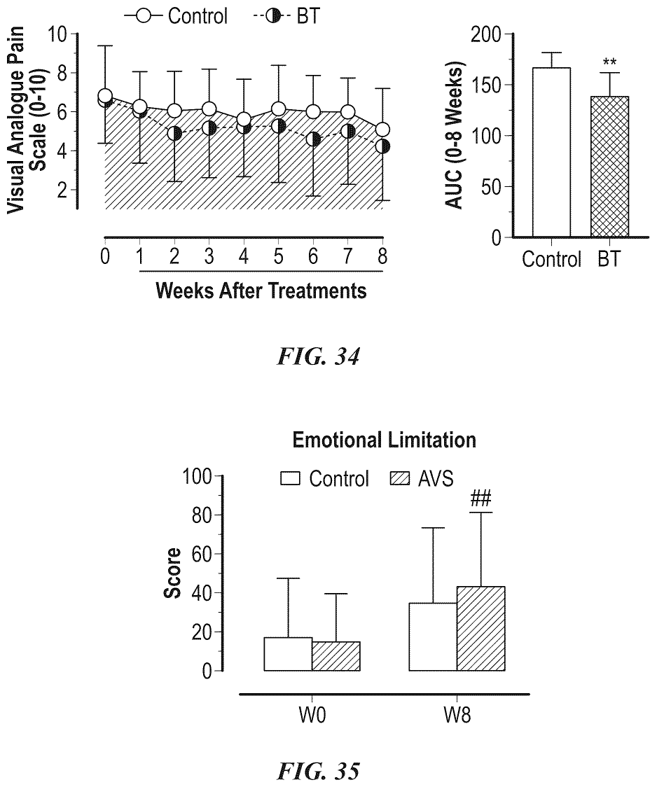

FIG. 33 and FIG. 34 depict two graphs showing results from medical outcome studies. The left graph of FIG. 33 shows limitations due to emotional aspects measured using the SF-36 survey, comparing scores between a placebo group and a BrainTap group at baseline (W4) and after 8 weeks (W8) of treatment. The right graph displays mental health scores measured using the SF-36 survey, comparing results between placebo and BrainTap groups at baseline and after 8 weeks of treatment. Both graphs include individual data points plotted over box plots showing the statistical distribution of scores, with error bars indicating the range of measurements. FIG. 34 provides a Visual Analog Scale (VAS) to evaluate pain after treatment for 8-weeks. A control group and a BrainTap (BT) treatment group were monitored, with the BrainTap group showing a more significant lowering of pain than the control group.

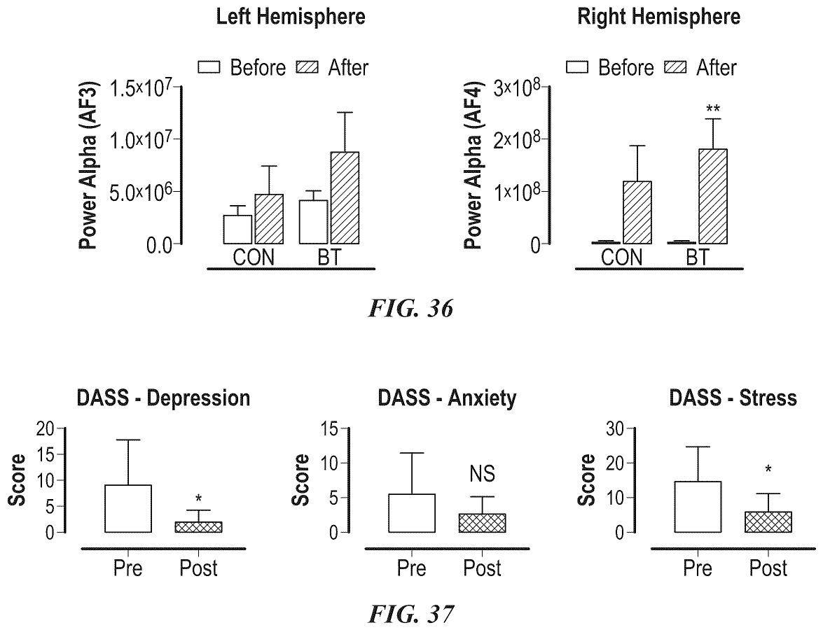

FIG. 35 and FIG. 36 show graphs illustrating emotional limitation scores and alpha wave activity measurements in brain hemispheres. FIG. 35 displays emotional limitation scores comparing control and AVS groups at timepoints W0 and W8. FIG. 36 shows alpha wave power measurements from electrodes AF3 and AF4 positioned over the left and right hemispheres respectively, comparing activity levels before and after treatment in control and brain training conditions.

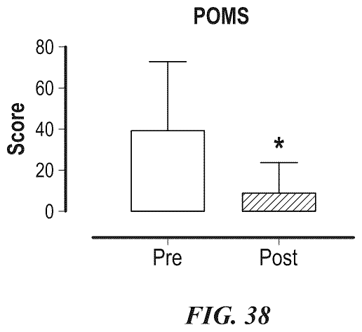

FIG. 37 and FIG. 38 present graphs showing results from psychological assessment scales. FIG. 37 displays Depression, Anxiety and Stress Scale (DASS-21) scores comparing pre- and post-intervention measurements across three categories: depression, anxiety, and stress. The depression scores show a statistically significant decrease from pre to post measurement, as indicated by an asterisk. The anxiety scores show a non-significant decrease from pre to post measurement, as indicated by “NS”. The stress scores demonstrate a statistically significant decrease from pre to post measurement, as indicated by an asterisk. FIG. 38 displays Profile of Mood States (POMS) scores comparing pre- and post-intervention measurements. The POMS scores show a statistically significant decrease from pre to post measurement, as indicated by an asterisk. Both graphs include error bars representing statistical variance in the measurements.

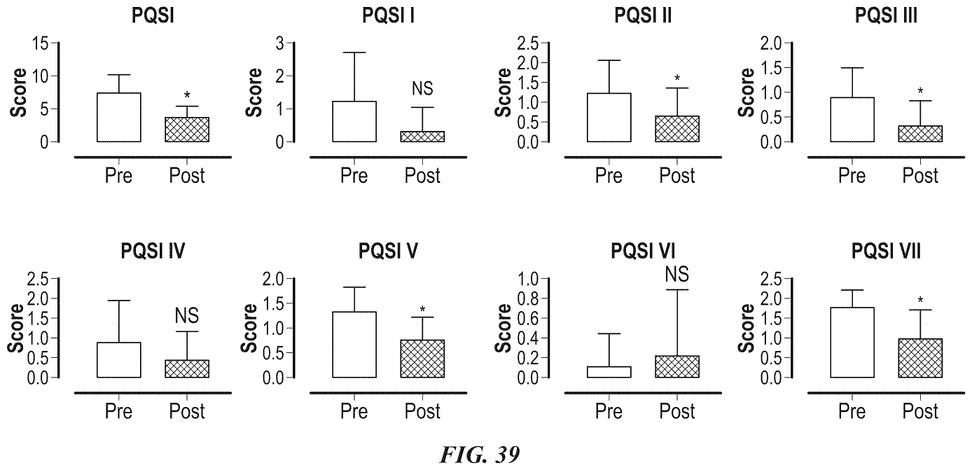

FIG. 39 illustrates a series of bar graphs showing Pittsburgh Quality of Sleep Index (PQSI) scores before and after an intervention. The graphs display eight different PQSI components measured at pre-intervention and post-intervention timepoints. The global PQSI score shows a decrease from pre to post measurements, with statistical significance indicated by an asterisk. Component 1 measuring subjective sleep quality demonstrates a non-significant decrease from pre to post measurements. Component 2 measuring sleep latency shows a non-significant reduction between timepoints. Component 3 measuring sleep duration exhibits a statistically significant decrease from pre to post measurements. Component 4 measuring sleep efficiency displays a non-significant reduction between timepoints. Component 5 measuring sleep disturbance shows a statistically significant decrease from pre to post measurements. Component 6 measuring use of sleep medication demonstrates a non-significant change between timepoints. Component 7 measuring daytime dysfunction exhibits a statistically significant decrease from pre to post measurements.

FIG. 40 presents a perspective view of a wearable neurostimulation device. The device comprises a curved headband structure that extends around the head, with integrated optical components positioned on both sides. The headband includes a visor portion that extends across the front and is designed to cover the user's eyes. The device incorporates circular housings integrated into the headband structure. These housings may contain optical components that can be used for delivering visual signals or photobiomodulation. The optical components are arranged in a symmetrical configuration on both sides of the headband. The device includes headphone components attached to the sides of the headband. The headphones feature circular ear cups connected to the main structure through adjustable mounting points. A black band extends across the top portion of the device, providing structural support and adjustability. The overall design integrates the visual stimulation components within the visor section while incorporating audio delivery capabilities through the headphone assemblies. The curved structure of the headband follows ergonomic contours to provide comfort during wear. The device demonstrates a modular construction that combines multiple stimulation modalities in a wearable form factor. The device features a two-tone color scheme with light and dark sections. Various electronic and optical components are visible through cutaway portions of the housing, revealing the internal architecture. The integration of these components allows for synchronized delivery of audio and visual stimuli to the user.

FIG. 41 illustrates a perspective rear view of a neurostimulation headset device. The device comprises a curved headband structure that wraps around the circumference of the head. The headband includes a visor portion that extends across the front and sides, designed to cover the eyes of a user. The device incorporates optical components housed within circular enclosures positioned along the inner curve of the headband. These circular housings contain light-emitting diode arrays with transparent or translucent covers for delivering visual signals. The headset includes headphone components positioned on either side, with circular ear cups connected to the visor through adjustable arms. The headphone components provide audio delivery capabilities while maintaining a streamlined integration with the overall headset design. The device features a modular construction that combines the visor, headband, and audio components into a unified wearable form factor. The curved design of the headband follows the natural contours of the head for improved fit and comfort during use. The rear view shows the internal component layout through a partially transparent or wireframe representation. Green-colored sections indicate possible locations of electronic components and circuit boards within the device structure. A mounting mechanism integrated into the headband allows for size adjustment to accommodate different head sizes.

As stated above, the neurostimulation system comprises at least a headset and a visor with an optional helmet that work in combination to deliver specific audio and visual stimuli. As shown in FIG. 40, the system includes a curved headband structure that extends around the head, with integrated optical components positioned on both sides. The headband incorporates a visor portion that extends across the front and is designed to cover the user's eyes.

The headset may provide audio signals such as binaural beats or isochronic tones. In some cases, the headset includes headphone components attached to the sides of the headband, as depicted in FIG. 40. The headphones may feature circular ear cups connected to the main structure through adjustable mounting points.

The visor may emit pulsed light at particular frequencies and wavelengths. As illustrated in FIG. 40, the visor section includes circular housings integrated into the headband structure.

These housings may contain optical components that can be used for delivering visual signals. The optical components may be arranged in a symmetrical configuration on both sides of the headband.

If a helmet is constructed, the helmet may deliver transcranial photobiomodulation. While not explicitly shown in FIG. 40 or 41, the helmet component may incorporate additional light-emitting elements designed to target specific regions of the brain.

FIG. 41 provides a rear view of the neurostimulation device, showing the curved headband structure that wraps around the circumference of the head. This view reveals the integration of the visor, headband, and audio components into a unified wearable form factor. The device may feature a modular construction that allows for the combination of multiple stimulation modalities.

In some cases, the audio and visual stimuli are precisely timed and synchronized to induce desired brainwave states. The neurostimulation system may generate specific brainwave patterns associated with relaxation, creativity, or deep sleep. By delivering carefully calibrated stimuli, the system may aim to modulate brainwave activity in a non-invasive manner.

The combination of auditory and visual entrainment provided by the headset and visor, along with the photobiomodulation capabilities of the helmet, may offer a comprehensive approach to neurostimulation. This integrated design may allow for simultaneous delivery of multiple forms of stimulation to potentially enhance the overall effectiveness of the therapy.

In some cases, the neurostimulation system may include a headset component designed to deliver audio signals to a user. As shown in FIG. 40, the headset may comprise headphone components attached to the sides of a curved headband structure. The headphones may feature circular ear cups connected to the main structure through adjustable mounting points, allowing for customized fit and comfort.

The headset component may be configured to generate and deliver specific audio signals, including binaural beats and isochronic tones. Binaural beats may be created when two slightly different frequencies are presented separately to each ear, resulting in the perception of a third tone at the frequency difference between the two original tones. Isochronic tones may involve a single tone that is turned on and off rapidly at regular intervals.

In some cases, the headset may be capable of producing audio signals within a frequency range of 18 to 0.5 Hz. This frequency range may be significant as it encompasses various brainwave states associated with different levels of consciousness and cognitive function. For example, frequencies near 18 Hz may correspond to beta waves associated with active, alert states, while frequencies approaching 0.5 Hz may align with delta waves typically observed during deep sleep or meditative states.

The headset component may utilize advanced audio processing techniques to generate precise binaural beats and isochronic tones within the specified frequency range. In some cases, the headset may incorporate digital signal processing capabilities to ensure accurate frequency production and timing of the audio signals.

As illustrated in FIG. 41, the headset component may be integrated into a larger neurostimulation device that includes a visor portion. The rear view shown in FIG. 41 demonstrates how the headphone components may be positioned in relation to the overall device structure, allowing for simultaneous delivery of audio and visual stimuli.

In some cases, the headset component may be designed to work in conjunction with other elements of the neurostimulation system, such as the visor and helmet components. The integration of these components may allow for synchronized delivery of audio, visual, and photobiomodulation stimuli to potentially enhance the overall effectiveness of the neurostimulation therapy.

The ability of the headset to deliver audio signals within the 18 to 0.5 Hz range may provide flexibility in targeting specific brainwave states. For instance, audio signals in the higher end of this range may be used to promote alertness or focus, while lower frequencies may be employed to induce relaxation or sleep. This versatility may allow the neurostimulation system to be adapted for various therapeutic applications or cognitive enhancement purposes.

In some cases, the neurostimulation system may include a visor component designed to deliver visual signals to a user. As shown in FIG. 40, the visor portion extends across the front of the headband structure and is designed to cover the user's eyes. The visor may incorporate circular housings integrated into the headband structure, which may contain optical components for delivering visual stimulation.

The visor component may be configured to emit pulsed light at specific frequencies and wavelengths. In some cases, the visor may utilize light-emitting diodes (LEDs) that emit light at a wavelength of 470 nanometers. This particular wavelength falls within the blue light spectrum and may be chosen for its potential effects on brain activity and circadian rhythms.

The LEDs in the visor may be designed to flicker at frequencies ranging from 18 Hz to 0.5 Hz. This frequency range encompasses various brainwave states associated with different levels of consciousness and cognitive function. For example, frequencies near 18 Hz may correspond to beta waves associated with active, alert states, while frequencies approaching 0.5 Hz may align with delta waves typically observed during deep sleep or meditative states.

In some cases, the flickering light emitted by the visor may be used to induce specific brainwave patterns through a process known as visual entrainment. This technique may involve synchronizing the brain's electrical activity with the frequency of the flickering light, potentially influencing the user's mental state or cognitive performance.

As illustrated in FIG. 41, the visor component may be integrated into the overall structure of the neurostimulation device. The rear view shown in FIG. 41 demonstrates how the visor portion wraps around the front and sides of the head, allowing for comprehensive visual stimulation coverage.

The visual signals delivered by the visor may work in conjunction with other elements of the neurostimulation system, such as the headset and helmet components. In some cases, the combination of visual stimulation from the visor and audio signals from the headset may provide a more potent effect than visual or audio stimulation alone.

The ability of the visor to deliver light at 470 nanometers and flicker within the 18 to 0.5 Hz range may offer flexibility in targeting specific brainwave states. For instance, higher flickering frequencies may be used to promote alertness or focus, while lower frequencies may be employed to induce relaxation or sleep. This versatility may allow the neurostimulation system to be adapted for various therapeutic applications or cognitive enhancement purposes.

In some cases, the neurostimulation system may include a helmet component designed to deliver transcranial photobiomodulation to a user. While not explicitly shown in FIG. 40 or 41, the helmet component may be integrated with the headset and visor components to provide a comprehensive neurostimulation device.

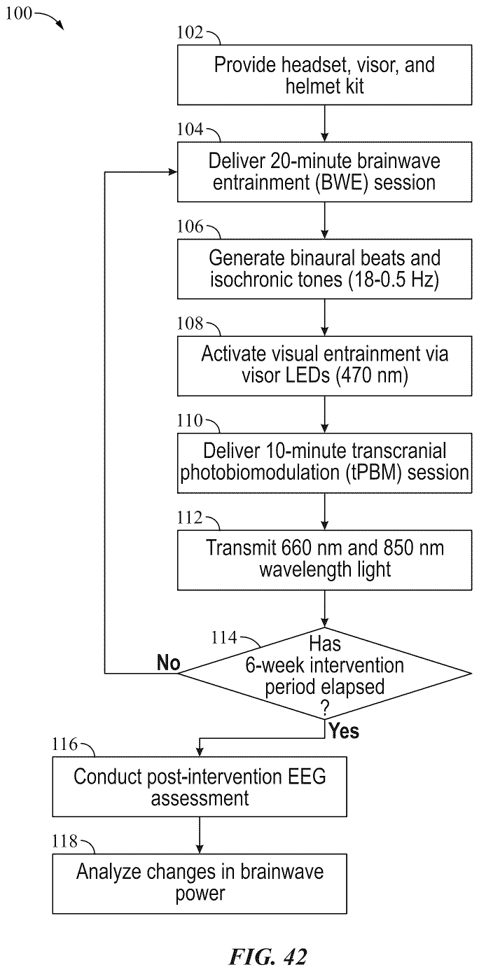

FIG. 42 illustrates a flowchart of a method 100 for delivering neurostimulation therapy. The method 100 begins at step 102, where a headset, visor, and helmet kit is provided to a user. The method 100 proceeds to step 104, where a 20-minute Brainwave Entrainment (BWE) session is delivered. Following this, at step 106, binaural beats and isochronic tones are generated within a frequency range of 18-0.5 Hz. At step 108, visual entrainment is activated via light-emitting diodes (LEDs) in the visor operating at 470 nm. The method 100 then moves to step 110, where a 10-minute Transcranial Photobiomodulation (tPBM) session is delivered. The method 100 continues to step 112, where light at wavelengths of 660 nm and 850 nm is transmitted. At step 114, the method 100 determines whether a 6-week intervention period has elapsed. If the period has not elapsed (No branch), the method 100 returns to earlier steps to continue the intervention. If the 6-week period has elapsed (Yes branch), the method 100 proceeds to step 116, where a post-intervention EEG assessment is conducted. The method 100 concludes at step 118 with analysis of changes in brainwave power. The flowchart depicts the sequence of steps for delivering combined BWE and tPBM therapy, including the timing of sessions, specific frequencies and wavelengths used, and assessment procedures.

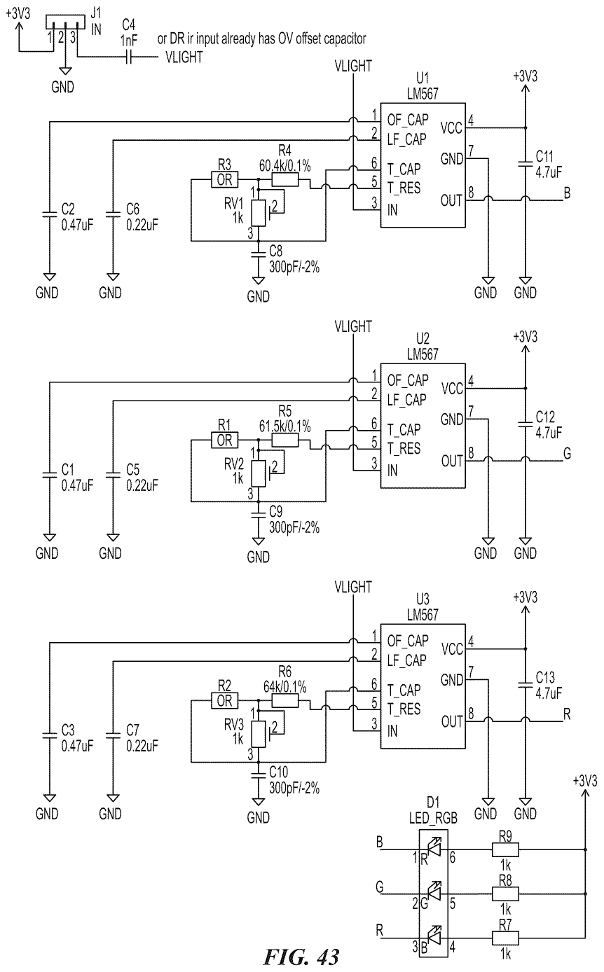

FIG. 43 illustrates a frequency detection and signal processing circuit with three parallel channels. The circuit includes a first frequency detector U1, a second frequency detector U2, and a third frequency detector U3 connected in parallel. An input coupling capacitor Cl is connected to first frequency detector U1. A filter capacitor C2 and timing capacitor C3 provide signal conditioning for first frequency detector U1. An output capacitor C4 is connected to the output of first frequency detector U1. A frequency tuning capacitor C5 and loop filter capacitor C6 establish frequency response characteristics for first frequency detector U1. A second input capacitor C7 is connected to second frequency detector U2. A second timing capacitor C8 provides timing control for second frequency detector U2. A second output capacitor C9 is connected to the output of second frequency detector U2. A baseline resistor R1, tuning resistor R2, and trim resistor R3 form a resistor network that sets operating parameters for the frequency detectors. The baseline resistor R1 is in the kiloohm range, tuning resistor R2 is in the hundreds of ohm range, and trim resistor R3 is in the hundred-thousands of ohms range. The frequency detectors U1, U2, and U3 are configured to detect distinct near-ultrasonic frequencies within a range of 18,000 to 20,000 Hz. Upon detection of their respective frequencies, each frequency detector activates a corresponding light channel. The parallel configuration of frequency detectors U1, U2, and U3 allows for simultaneous detection of multiple frequencies from a shared audio input signal. The capacitors C1, C2, C3, C4, C5, C6, C7, C8, and C9 are sized larger than conventional designs to enhance circuit stability. The resistor network formed by R1, R2, and R3 provides frequency tuning while compensating for component tolerances.

More specifically in order to more fully explain the circuit diagram of FIG. 43, the first frequency detector U1 plays an important role in the frequency detection and signal processing circuit, designed to identify specific near-ultrasonic frequencies within the range of 18,000 to 20,000 Hz. U1 is configured to process audio signals received from a shared input, which is typically conditioned by a preamplifier. The input coupling capacitor C1 is connected to U1, serving to couple the input signal to the frequency detector, ensuring that the signal is appropriately conditioned for processing. This setup allows Ul to filter and detect the desired frequency range, which is necessary for activating corresponding light channels in the neurostimulation system.

The second frequency detector U2 operates in parallel with U1, sharing the same audio input signal. U2 is similarly configured to detect distinct frequencies within the specified range, contributing to the system's ability to process multiple frequencies simultaneously. The second input capacitor C7 is connected to U2, ensuring that the input signal is properly coupled for frequency detection. U2's configuration includes a second timing capacitor C8, which sets the timing characteristics necessary for accurate frequency detection. The second output capacitor C9 is responsible for coupling the processed signal to subsequent stages, enabling the activation of light channels based on the detected frequencies.

The third frequency detector U3 complements U1 and U2 by providing an additional channel for frequency detection. U3 is equipped with similar components, including a baseline resistor R1, a tuning resistor R2, and a trim resistor R3, which form a tuning network that provides precise control over the frequency response. This network allows U3 to be finely tuned to detect specific frequencies with high accuracy. The configuration of U3 ensures that the detector can operate in harmony with U1 and U2, allowing the system to detect and process multiple frequencies simultaneously, thereby enhancing the overall functionality of the neurostimulation system.

The input coupling capacitor C1 is important in the frequency detection circuit by ensuring that the audio signal is suitably coupled to the first frequency detector U1. C1 is oversized by a factor of approximately 2.1 compared to conventional designs, which enhances the stability and performance of the circuit across variations in temperature and component tolerances. This design choice helps maintain consistent signal quality, which is necessary for accurate frequency detection and subsequent light channel activation.

The filter capacitor C2 is connected to the first frequency detector U1 and serves to filter out unwanted frequencies, allowing only the desired frequency range to be processed. This filtering capability is important for ensuring that the frequency detector can accurately identify the target frequencies without interference from extraneous signals. C2's role in the circuit is to maintain the integrity of the frequency detection process, which is essential to the operation of the neurostimulation system.

The timing capacitor C3 is used to set the timing characteristics of the first frequency detector U1. By establishing the timing parameters, C3 ensures that U1 can accurately detect the desired frequencies within the specified range. The precise timing control provided by C3 plays a significant role in synchronizing the frequency detection process with the activation of light channels, thereby enabling the neurostimulation system to deliver targeted therapy.

The output capacitor C4 facilitates the transfer of the processed signal from the first frequency detector U1 to the subsequent stage of the circuit. C4 helps in transmitting the signal with minimal loss, preserving the quality and integrity of the frequency detection process. This coupling is important for activating the corresponding light channels based on the detected frequencies, which serves an important function of the neurostimulation system.

The frequency tuning capacitor C5 is used to fine-tune the frequency response of the first frequency detector U1. By adjusting the frequency characteristics, C5 allows for precise frequency detection, which is necessary for the accurate operation of the neurostimulation system. The ability to fine-tune the frequency response ensures that the system can adapt to different audio inputs and maintain high detection accuracy.

The loop filter capacitor C6 is connected to the second frequency detector U2 and plays a role in stabilizing the frequency detection process. By providing a stable frequency response, C6 ensures that U2 can accurately process the input signal and detect the desired frequencies. This stability is important for maintaining the overall performance of the frequency detection circuit and ensuring reliable operation of the neurostimulation system.

The second input capacitor C7 is connected to the second frequency detector U2, serving a similar function as C1 by coupling the input signal to the frequency detector. C7 ensures that the signal is properly conditioned for processing, which is necessary for accurate frequency detection. The role of C7 in the circuit is to maintain the quality of the input signal, enabling U2 to perform the frequency detection function effectively.

The second timing capacitor C8 is used to set the timing characteristics of the second frequency detector U2. By establishing the timing parameters, C8 ensures that U2 can accurately detect the desired frequencies within the specified range. The precise timing control provided by C8 is crucial for synchronizing the frequency detection process with the activation of light channels, thereby enabling the neurostimulation system to deliver targeted therapy.

The second output capacitor C9 facilitates the transfer of the processed signal from the second frequency detector U2 to the subsequent stage of the circuit. C9 helps to transmit the signal with minimal loss, preserving the quality and integrity of the frequency detection process. This coupling is important for activating the corresponding light channels based on the detected frequencies, which serves an important function of the neurostimulation system.

The baseline resistor R1 is part of a tuning network that provides precise control over the frequency response of each frequency detector. R1 is in the kiloohm range and is used to set the baseline frequency for detection. This resistor plays a significant role in ensuring that the frequency detectors can accurately identify the target frequencies within the specified range, contributing to the overall functionality of the neurostimulation system.

The tuning resistor R2 is also part of the tuning network and is used to adjust the frequency response of each frequency detector. R2 is in the hundreds of ohm range and provides the necessary tuning capability to ensure accurate frequency detection. The role of R2 in the circuit is to allow for fine adjustments to the frequency response, enabling the system to adapt to different audio inputs and maintain high detection accuracy.

The trim resistor R3 is the final component of the tuning network and is used to trim the frequency response of each frequency detector. R3 is in the hundred-thousands of ohms range and provides the necessary resistance to fine-tune the frequency characteristics. The inclusion of R3 in the circuit ensures that the frequency detectors can be precisely calibrated to detect the desired frequencies, enhancing the overall performance of the neurostimulation system.

A number of implementations have been described. Nevertheless, it will be understood that various modifications may be made without departing from the spirit and scope of the disclosure. Accordingly, other implementations are within the scope of the following claims.

We claim:

1. A neurostimulation device(s), comprising:
at least a headset and/or headset and visor configured to deliver audio signals including binaural beats and isochronic tones in a frequency range from 18 to 0.5 Hz;
wherein said visor is configured to deliver visual signals via light-emitting diodes emitting light at 470 nanometers and flickering at frequencies between 18 and 0.5 Hz;
and optionally a helmet or said headset and/or visor are configured to deliver transcranial photobiomodulation via light at 660 nm and 850 nm wavelengths with a total irradiance of 1000 m W/cm2 per minute; and
a controller operatively connected to the headset, visor, and optional helmet, said controller configured to synchronize and control delivery of said audio signals, visual signals, and photobiomodulation to alter brainwave activity in a user.

2. The neurostimulation device(s) of claim 1, wherein said controller is configured to adjust a frequency of said audio signals and visual signals dynamically during a therapy session.

3. The neurostimulation device(s) of claim 2, wherein said controller is configured to transition said frequency of said audio signals and visual signals from higher frequencies to lower frequencies over a course of a therapy session.

4. The neurostimulation device(s) of claim 1, wherein either said headset and/or visor or an optional helmet comprises a plurality of light-emitting diode arrays distributed across its inner surface to provide homogeneous light distribution.

5. The neurostimulation device(s) of claim 4, wherein said light-emitting diode arrays in said headset and/or visor or an optional helmet comprise 100 LEDs emitting light at 660 nm and 100 LEDs emitting light at 850 nm.

6. The neurostimulation device(s) of claim 1, further comprising an electroencephalogram (EEG) measurement device configured to measure brainwave activity before and after a neurostimulation therapy session.

7. The neurostimulation device(s) of claim 6, wherein said controller is configured to adjust delivery of audio signals, visual signals, and photobiomodulation based on feedback from said EEG measurement device.

8. A method for delivering neurostimulation therapy, comprising:
providing a headset, and a visor, wherein said headset and visor can be a singular unit; and
optionally a helmet to a user;
delivering a Brainwave Entrainment (BWE) session by generating audio signals including binaural beats and isochronic tones in a frequency range from 18 to 0.5 Hz through said headset and activating visual signals via light-emitting diodes emitting light at 470 nanometers and flickering at frequencies between 18 and 0.5 Hz through said visor;
delivering a Transcranial Photobiomodulation (tPBM) session by transmitting light at 660 nm and 850 nm wavelengths through the helmet with a total irradiance of 1000 mW/cm2 per minute; and
synchronizing said delivery of said BWE session and said tPBM session to alter brainwave activity in one or more users.

9. The method of claim 8, wherein the BWE session lasts for 20 minutes and said tPBM session lasts for 10 minutes.

10. The method of claim 9, further comprising repeating said BWE session and tPBM session daily over a 6-week intervention period.

11. The method of claim 10, further comprising conducting an electroencephalogram (EEG) assessment before and after said 6-week intervention period to measure changes in brainwave activity.

12. The method of claim 11, wherein the EEG assessment comprises a 2-minute eyes opened measurement followed by a 2-minute eyes closed measurement.

13. The method of claim 12, wherein the EEG assessment measures brainwave power across 14 channels corresponding to different regions of a human brain.

14. The method of claim 13, further comprising analyzing changes in brainwave power across delta, theta, alpha, beta, and gamma frequency bands between pre-intervention and post-intervention EEG assessments.

15. A circuit for detecting audio frequencies and activating light channels, comprising:
a plurality of frequency detectors, each configured to detect a distinct near-ultrasonic frequency within a range of 18,000 to 20,000 Hz;
a Bluetooth module configured to receive an audio signal;
a preamplifier coupled to the Bluetooth module for signal conditioning; and
a plurality of light activation channels, each triggered by an output of a corresponding frequency detector.

16. The circuit of claim 15, wherein the plurality of frequency detectors comprises three LM567 integrated circuits, each configured to detect a distinct near-ultrasonic frequency.

17. The circuit of claim 16, wherein each LM567 integrated circuit is configured with a combination of passive components that provide an ability to tune a frequency response and bandwidth.

18. The circuit of claim 17, wherein said passive components for each LM567 integrated circuit(s) include a baseline resistor in a kiloohm range, a tuning resistor in a hundreds of ohm range, and a trim resistor in parallel that provides resistance in a range of hundred-thousands of ohms or (kΩ).

19. The circuit of claim 18, wherein capacitors connected to said LM567 integrated circuits are oversized by a factor of approximately 2.1 compared to conventional designs.

20. The circuit of claim 19, further comprising a volume control implemented using a two-channel potentiometer placed between a preamplifier and an audio transducer.

21. A neurostimulation system, comprising: a headset configured to deliver audio signals including binaural beats and isochronic tones in a frequency range from 18 to 0.5 Hz; a visor configured to deliver visual signals via light-emitting diodes emitting light at 470 nanometers and flickering at frequencies between 18 and 0.5 Hz wherein said headset and visor can be a singular unit; and optionally a helmet, wherein at least said system includes said headset and said visor configured to deliver transcranial photobiomodulation, with said headset, visor and optional helmet comprising: a plurality of light-emitting diode arrays arranged along an inner surface for homogeneous light distribution, the arrays configured to emit light at 660 nm and 850 nm wavelengths to deliver a total irradiance of 1000 mW/cm² per minute; and a controller operatively connected to said headset, visor, and optional helmet, said controller configured to synchronize and control delivery of audio signals, visual signals, and photobiomodulation that alters brainwave activity in a user.

22. The neurostimulation system of claim 21, wherein said controller is configured to adjust a frequency of audio signals and visual signals dynamically during a therapy session.

23. The neurostimulation system of claim 21, wherein said controller is configured to transition said frequency of audio signals and visual signals from higher frequencies to lower frequencies over a course of a therapy session.

24. The neurostimulation system of claim 21, wherein said headset, visor and optional helmet includes multiple light-emitting diode arrays arranged along an inner surface or all or either of said headset, visor and optional helmet that provides uniform light distribution.

25. The neurostimulation system of claim 24, wherein said light-emitting diode arrays comprise 100 LEDs emitting light at 660 nm and 100 LEDs emitting light at 850 nm and further comprising an electroencephalogram (EEG) measurement device configured to measure brainwave activity before and after a neurostimulation therapy session.

26. The neurostimulation system of claim 25, wherein said controller is configured to adjust delivery of audio signals, visual signals, and photobiomodulation based on feedback from said EEG measurement device.

27. A method for using the system of claim 21 by delivering neurostimulation therapy, comprising: providing at least one of a group consisting of a headset, a visor, and an optional helmet to a user wherein said headset and visor can be a singular unit; delivering a brainwave entrainment session by generating audio signals including binaural beats and isochronic tones in a frequency range from 18 to 0.5 Hz through said one of a group including said headset and activating visual signals via light-emitting diodes emitting light at 470 nanometers and flickering at frequencies between 18 and 0.5 Hz through said visor; delivering a transcranial photobiomodulation session by transmitting light at 660 nm and 850 nm wavelengths through said helmet with a total irradiance of 1000 mW/cm² per minute; and synchronizing delivery of said audio signals, visual signals, and photobiomodulation changing brainwave activity of said user.

28. The system of claim 27, further comprising measuring brainwave activity using an electroencephalogram (EEG) device.

29. The system of claim 28, further comprising adjusting delivery of audio signals, visual signals, and photo biomodulation based on measured brainwave activity.

30. The system of claim 29, wherein measured brainwave activity is provided as a feedback loop that is interpreted and used to control additional audio signals, visual signals and biomodulation of said signals sent to said user.
